(19) United States
(12) Patent Application Publication
(10) Pub. No.: US 2006/0116602 A1
(43) Pub. Date: Jun. 1, 2006

(54) Medical sensing device and system

(76) Inventor: Dana Andrew Alden, Clarendon Hills, IL (US)

Correspondence Address: Michael Pruden, 1000 Allanson Road, Mundelein, IL, 60060, US

(21) Appl. No.: 11/002,327

(22) Filed: Dec. 1, 2004

(51) Int. Cl. A61B 5/00; A61B 5/02

(52) U.S. Cl. 600/561; 128/903; 600/486

(57) ABSTRACT

The present invention relates to a medical device comprising (i) a sensing module including a housing that is generally cylindrical in shape; (ii) a sensing element located within the housing; (iii) a processing module electrically coupled to the sensing element that includes an analog-to-digital converter that is electrically connected to a microcontroller.

### FIELD OF THE INVENTION

This invention relates to medical sensors and software associated therewith.

### BACKGROUND OF THE INVENTION

Sensors have been used in the medical field. For example, an Intra-Compartmental Pressure Monitor System manufactured by Stryker International utilizes a syringe coupled to a side ported 18 gauge needle and a diaphragm. The syringe is filled with a sterile sodium chloride solution while the diaphragm separates the needle from the syringe. The needle is inserted into the patient and the sodium chloride solution within the syringe is pushed into the needle while a one-way valve prevents backflow of the sodium chloride solution into the syringe. Pressure within the patient's muscle compartment causes the sodium chloride solution within the needle to exert pressure on the diaphragm. The pressure exerted on the diaphragm is then measured.

There are problems, however, inherent in the foregoing system. Pressure within the muscle compartment is measured indirectly; compartmental pressure causes the sodium chloride solution within the needle to exert pressure on a diaphragm that is located adjacent to a syringe outside the muscle compartment. A direct measurement via a diaphragm that is inserted into the muscle compartment represents a more direct and more accurate method.

Pressure reading using the above-described Intra-Compartmental Pressure Monitor System can be erroneous. The side port on the needle can be occluded thereby preventing the fluid within patient's muscle compartment from forcing the sodium chloride solution up the needle to the diaphragm. Bubbles within the system also cause inaccuracies as compartmental fluids compress the bubbles rather than force the sodium chloride solution up the needle to the diaphragm. A leaky connection between the needle and the syringe also causes inaccuracies as fluid is forced out of the needle, rather than against the diaphragm. Furthermore, if a heparinized saline solution is used to flush the needle, bleeding from the needle insertion may falsely elevate local tissue pressure.

The present invention is directed to overcoming these and other disadvantages inherent in previous medical sensor systems.

### SUMMARY OF THE INVENTION

The scope of the present invention is defined solely by the appended claims, and is not affected to any degree by the statements within this summary. Briefly stated, a pressure sensor embodying features of the present invention comprises (i) a sensing module including a housing that is generally cylindrical in shape; (ii) a sensing element located within the housing; (iii) a processing module electrically coupled to the sensing element that includes an analog-to-digital converter that is electrically connected to a microcontroller.

### BRIEF DESCRIPTION OF THE DRAWINGS

FIG. 1 depicts a medical device including a sensing module, a computer and a server.

FIG. 2 depicts a view of the outside of the housing of the sensing component.

FIG. 3 depicts a cross-sectional view of the housing of the sensing component.

FIG. 4 depicts the processing module for a medical device.

FIG. 5 depicts a view of the outside of the housing of the sensing component.

FIG. 6 depicts the housing of the sensing component being inserted into a muscle compartment.

FIG. 7 depicts the housing being flexed at a 90° angle.

FIG. 8 depicts the housing that includes a plastic and a sensing element located within a protective medium

FIG. 9 depicts a sensing element provided with a plurality of layers that form a sealed cavity.

FIG. 10 depicts a sensing element provided with a piezoresistive element, contacts, and conducting material that electrically couples the sensing element to the processing and communications modules.

FIG. 11 depicts a sensing element provided with a plurality of layers that form an unsealed cavity.

FIG. 12 depicts a wafer.

FIG. 13 depicts a wafer provided with an epitaxial layer and intermediate layers.

FIG. 14 depicts a wafer provided with an epitaxial layer, intermediate layers, and a first phot-resist layer.

FIG. 15 depicts a wafer provided with an epitaxial layer, intermediate layers, a first phot-resist layer, and an etchant.

FIG. 16 depicts a wafer provided with an epitaxial layer with a first P diffusion and intermediate layers.

FIG. 17 depicts a wafer provided with an epitaxial layer with a first P diffusion, intermediate layers, and a second photo-resist layer.

FIG. 18 depicts a wafer provided with an epitaxial layer with a first P diffusion, both of which have been etched.

FIG. 19 depicts a wafer provided with an epitaxial layer with a first P diffusion and a second P diffusion.

FIG. 20 depicts a wafer provided with an epitaxial layer with first and second P diffusions and a third photo-resist layer.

FIG. 21 depicts a wafer provided with an epitaxial layer that has been etched for conducting material and further including first and second P diffusions.

FIG. 22 depicts a wafer provided with an epitaxial layer, first and second P diffusions, and conducting material.

FIG. 23 depicts a wafer provided with an epitaxial layer, first and second P diffusions, conducting material, and a fourth photo-resist layer.

FIG. 24 depicts a wafer provided with an epitaxial layer, first and second P diffusions, conducting material, and a plurality of contacts.

FIG. 25 depicts a wafer provided with an epitaxial layer, first and second P diffusions, conducting material, a plurality of contacts, and a fifth photo-resist layer.

FIG. 26 depicts a wafer provided with an epitaxial layer, first and second P diffusions, conducting material, a plurality of contacts, a pocket, and a fifth photo-resist layer.

FIG. 27 depicts a wafer provided with an epitaxial layer, first and second P diffusions, conducting material, a plurality of contacts, and a pocket after the fifth photo-resist layer has been removed.

FIG. 28 depicts a wafer provided with an epitaxial layer, first and second P diffusions, conducting material, a plurality of contacts, and a pocket bonded to a second layer to form an unsealed cavity.

FIG. 29 depicts a sensing element provided with a base, an emitter, a collector, a buried layer, a resistive element and conducting material that electrically couples the sensing element to a processing module.

FIG. 30 depicts a sensing element provided with a plurality of resistive elements, a diaphragm, a bond pad, and leads from the resistive elements.

FIG. 31 depicts a wireless module.

FIG. 32 depicts the circuit diagram of an integrated controller.

FIG. 33 depicts diagrammatically the integrated controller within a wireless module.

FIG. 34 depicts a media access controller.

FIG. 35 depicts a transceiver control unit.

FIG. 36 depicts the process flow of an embodiment of the medical device.

FIG. 37 depicts the process flow for an acquisition routine.

FIG. 38 depicts in greater detail the process flow of an embodiment of the medical device.

FIG. 39 depicts a handheld communications device receiving a text message.

FIG. 40 depicts an alert in the form of an e-mail.

FIG. 41 depicts a cross-sectional view of a sterilizer.

FIG. 42 depicts a cross-sectional view of a retaining device.

FIG. 43 depicts a cross-sectional view of an alternative sterilizer.

FIG. 44 depicts a cross-sectional view of an alternative retaining device.

FIG. 45 depicts a cross-sectional view of a sterilizer with the housing of a sensing module including a low profile processing module that is also shown cross-sectionally.

FIG. 46 depicts a cross-sectional view of a sterilizer with the housing of a sensing module including a low profile processing module that is not shown cross-sectionally.

### DETAILED DESCRIPTION OF THE PRESENTLY PREFERRED EMBODIMENTS

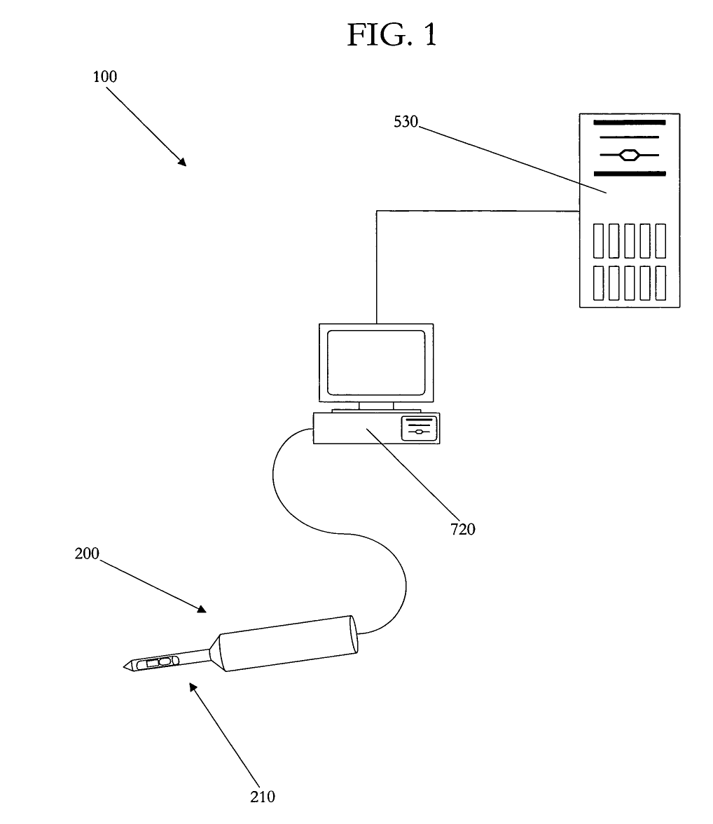

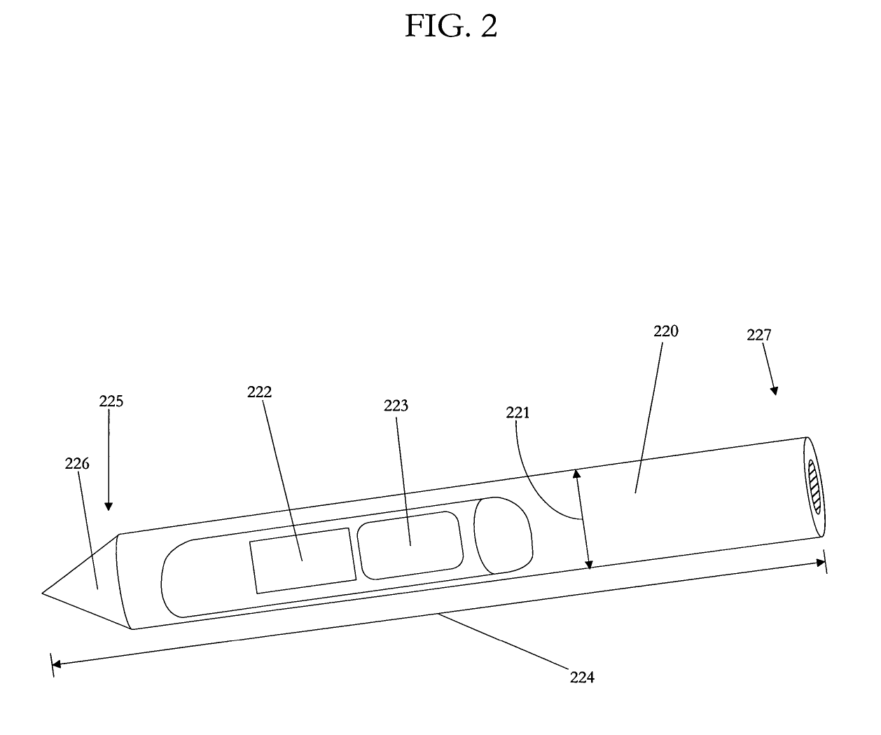

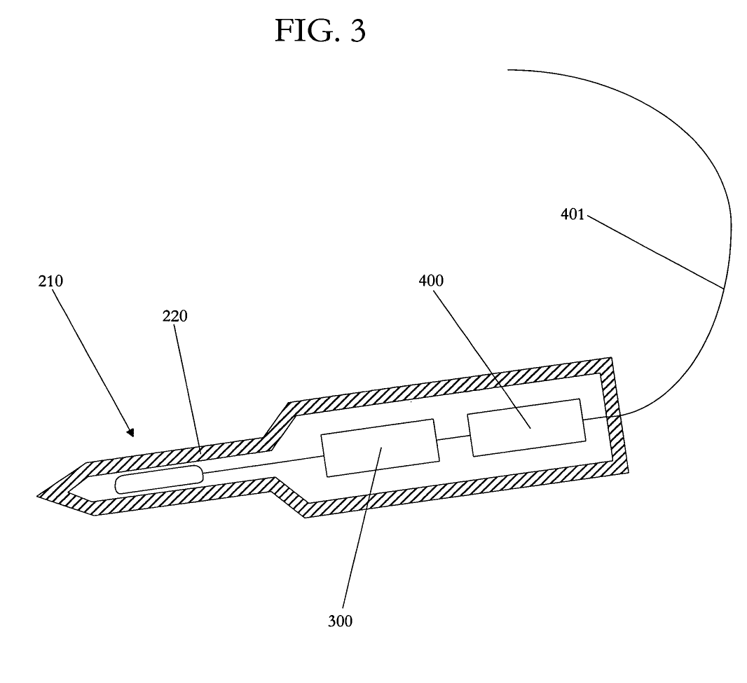

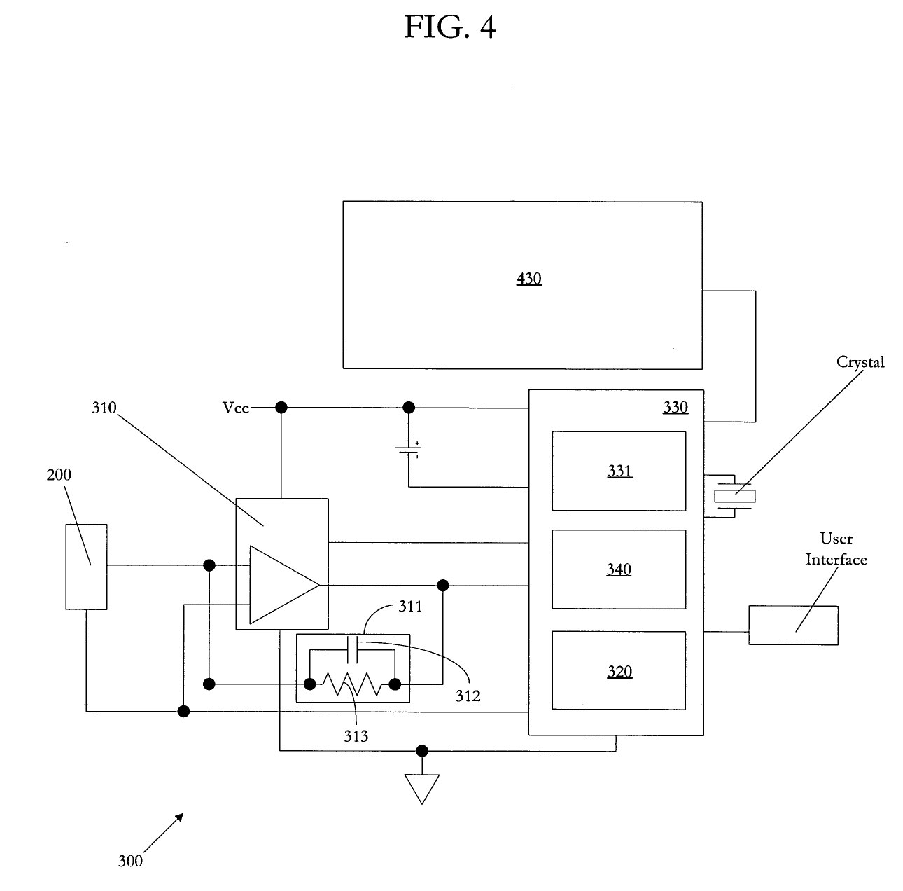

FIG. 1 depicts a presently preferred embodiment of the medical device 100. As shown therein, the medical device 100 is provided with a sensing module 200 that preferably includes a sensing component 210 as depicted in FIG. 2. The medical device 100 is also provided with a processing module 300 as shown in FIG. 3. FIG. 4 depicts the processing module 300 in greater detail. As shown therein, the processing module 300 includes an operational amplifier 310, an analog-to-digital converter 320, and a microcontroller 330. Additionally, as depicted in FIG. 3, the medical device 100 includes a communications module 400 and a communications link 401, such as a cable or a radio transmission, so that readings from the sensing module 200 are communicated to medical professionals and care-givers.

In the preferred embodiment, the sensing component 210 is a pressure sensor. As shown in FIG. 2, the sensing component 210 is provided with a housing 220 that is a hollow shaft and generally cylindrical in shape. Alternatively, the housing 220 is frustoconical in shape. In another alternative embodiment, the housing 220 is polygonal in cross section. Turning back to FIG. 2, the housing 220 is provided with a housing diameter 221 that is in the preferred range of 0.355 and 1.2 millimeters. The preferred embodiment depicted in FIG. 2 is provided with a housing diameter 221 of 0.355 millimeters; however, in alternative embodiments, the housing diameter 221 is increased up to 4 millimeters.

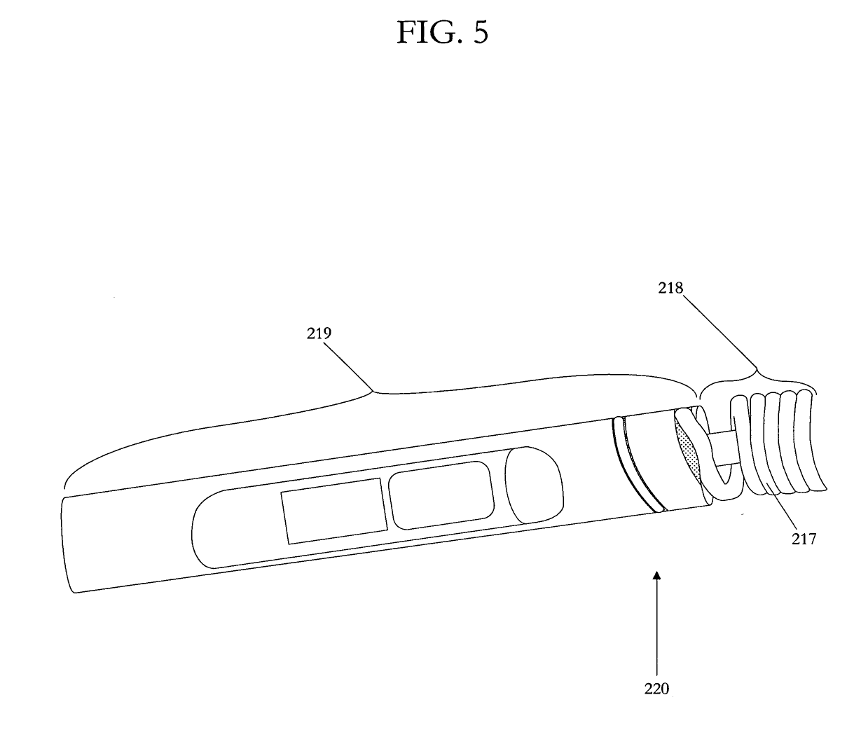

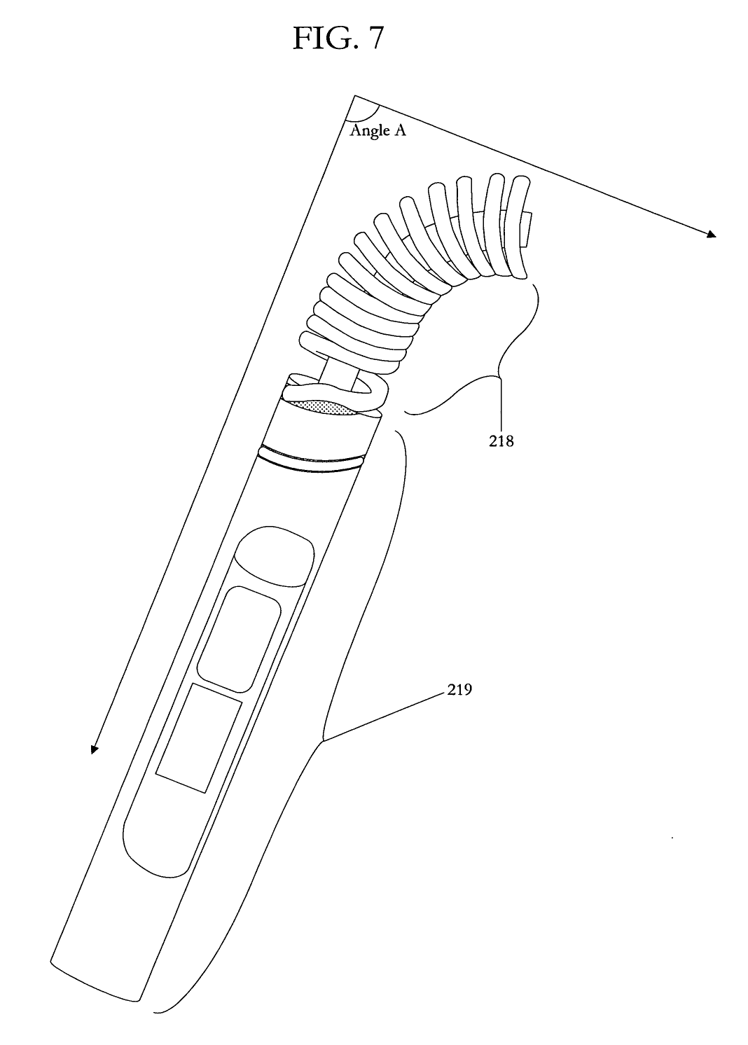

Referring now to FIG. 5, an alternative embodiment of the housing is shown. As depicted therein, the housing 220 includes a die-containing section 219 and a flexible section 218 that is provided with a helical portion 217. As shown in FIG. 7, the flexible section 218 is configured to flex so that the die-containing section 219 is positioned at an Angle A that measures 90°.

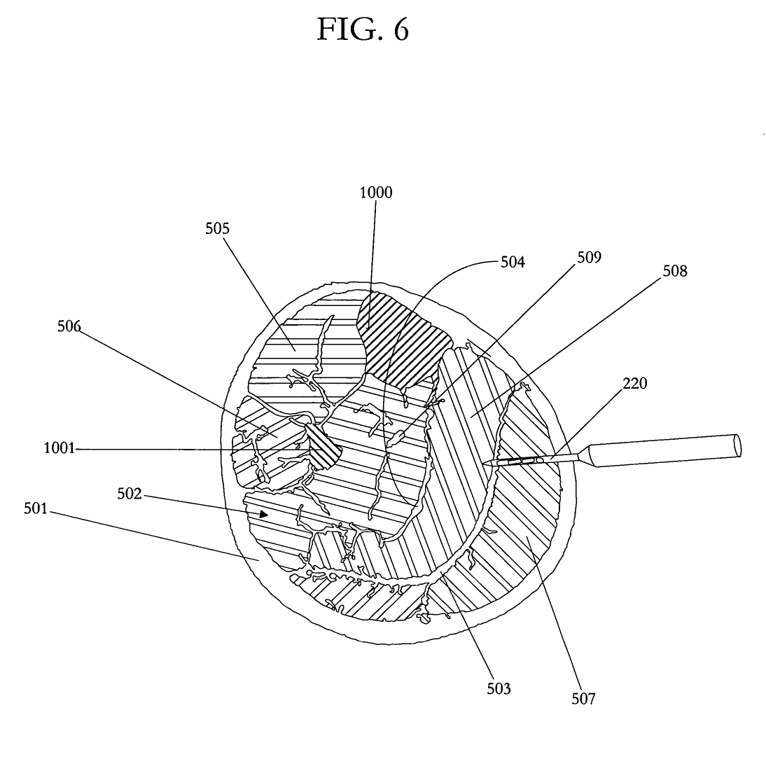

Referring now to FIG. 6, the housing 220 of the preferred embodiment is fabricated from a material that withstands the stresses of being inserted through the layers of the dermis and the epidermis 501, through muscle 502, through fat 509, and through at least one fascial layer 503. For ease of explanation, FIG. 6 depicts the muscle compartments around the tibia 1000 and the fibula 1001; however, the present invention is used in muscle compartments throughout the body, such as arms, forearms, hands, buttocks, thighs, etc. The dermis and epidermis 501, muscle 502, and a plurality of the fascial layers 503, 504 are shown in FIG. 6, as well as the anterior compartment 505, the lateral compartment 506, the superficial posterior compartment 507, and the deep posterior compartment 508. The housing 220 is shown penetrating through the fascial layer 503 and reaching the deep posterior compartment 508.

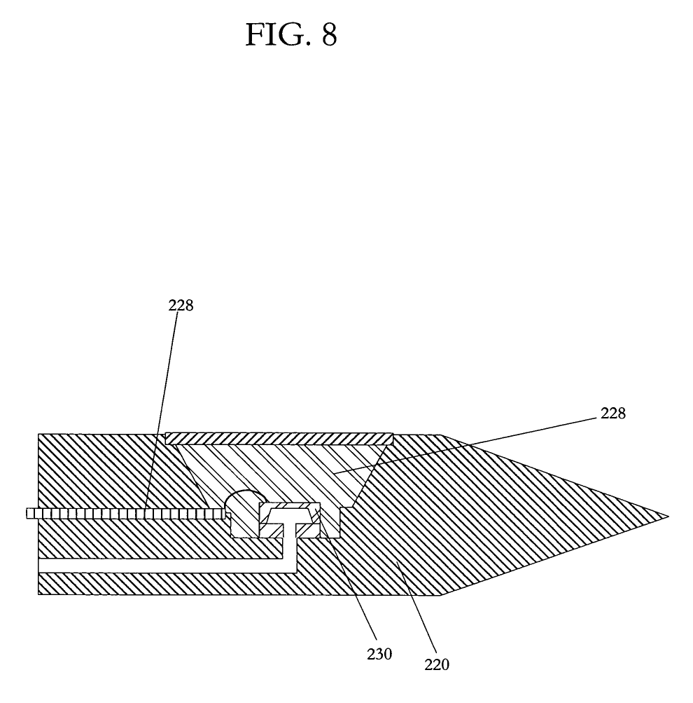

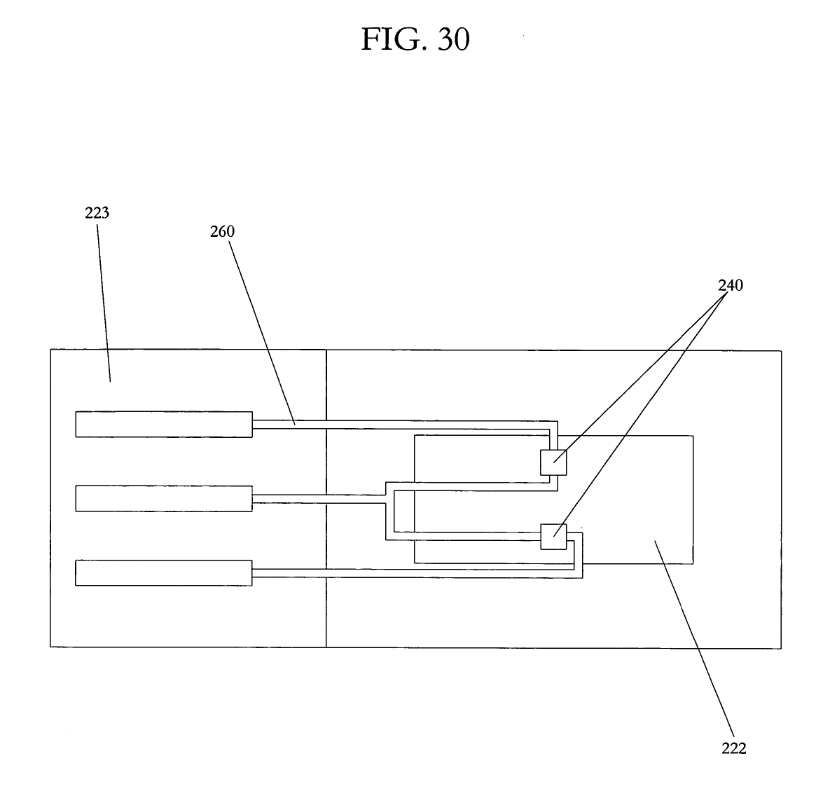

The housing 220 is fabricated from a metal, preferably stainless steel. In an alternative embodiment, as depicted in FIG. 8, the housing 220 is fabricated from an epoxy or plastic, such as a thermoplastic. In another alternative embodiment, the housing 220 is fabricated from both a plastic, such as a thermoplastic, and a metal, such as a stainless steel. In yet another alternative embodiment, the housing 220 is fabricated from titanium. The housing 220 is configured to allow a diaphragm 222 (shown in FIG. 30) to deflect in response to pressure. The housing 220 is also configured to accommodate a lead connecting area 223 (shown in FIG. 30).

Referring now to FIG. 6, the housing 220 is configured to be inserted into a compartment, such as the muscle compartment 500 of a patient. In the preferred embodiment, the housing 220 is configured to be inserted through the layers of the dermis 501, through muscle 502, and through the fascia 503 to reach the muscle compartment 500. As shown in FIG. 2 and FIG. 6, the housing 220 is provided with a housing length 224 that is dimensioned so that the diaphragm 222 is capable of being inserted through the layers of the dermis 501, through muscle 502, and through the fascia 503 and locating within the muscle compartment 500. In the preferred embodiment, the housing length 224 is 4 inches; however, in an alternative embodiment, the housing length 224 is less than 4 inches, such as between 1 and 4 inches.

The housing 220 is provided with a first end 225 and a second end 227. The first end 225 is configured to reach the muscle compartment 500 through piercing. The first end 225 is shaped to pierce through the layers of the dermis 501, through muscle 502, and through the fascia 503. As shown in FIG. 2, the housing 220 is provided with a tapering shape so that a piercing element 226 is located at the first end 225.

Referring now to FIG. 8, the sensing element 230 is a pressure sensor that is located within the housing 220. Advantageously, the sensing element 230 is located within a protective medium 228. Preferably, the protective medium 228 is a biocompatible material, such as a vulcanized rubber or a room temperature vulcanized rubber (referred to as “RTV”). Advantageously, the protective medium 228 includes a silicon, such as, for example, a silicon rubber. Alternatively, the protective medium 228 is a silicon gel. In another alternative embodiment, the protective medium 228 is an oil. In the embodiment depicted in FIG. 2, the pressure sensing element 230 is located within silicon rubber.

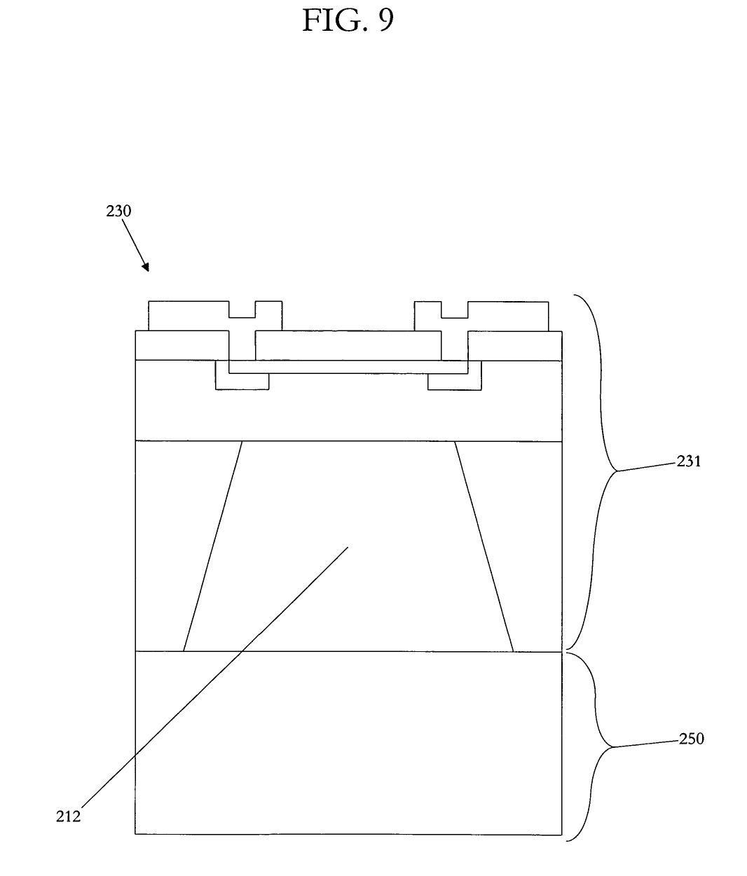

FIG. 9 depicts the presently preferred sensing element 230. As shown therein, the sensing element 230 is provided with a first layer 231 that includes a crystalline structure. Preferably, the first layer 231 includes a silicon. Alternatively, the first layer 231 includes a quartz. In another alternative embodiment, the first layer 231 includes a gallium arsenide. In yet another alternative embodiment, the first layer 231 includes a germanium.

The sensing element 230 is also provided with a second layer 250. In the preferred embodiment, the second layer 250 includes a glass, advantageously a glass that includes sodium, such as Pyrex 7740 glass. According to one aspect of the present invention, the glass is a borate glass, such as a borosilicate glass. According to another aspect, the glass includes lead. According to yet another aspect of the present invention, the glass includes zinc. In an alternative embodiment, the second layer 250 includes a silicon.

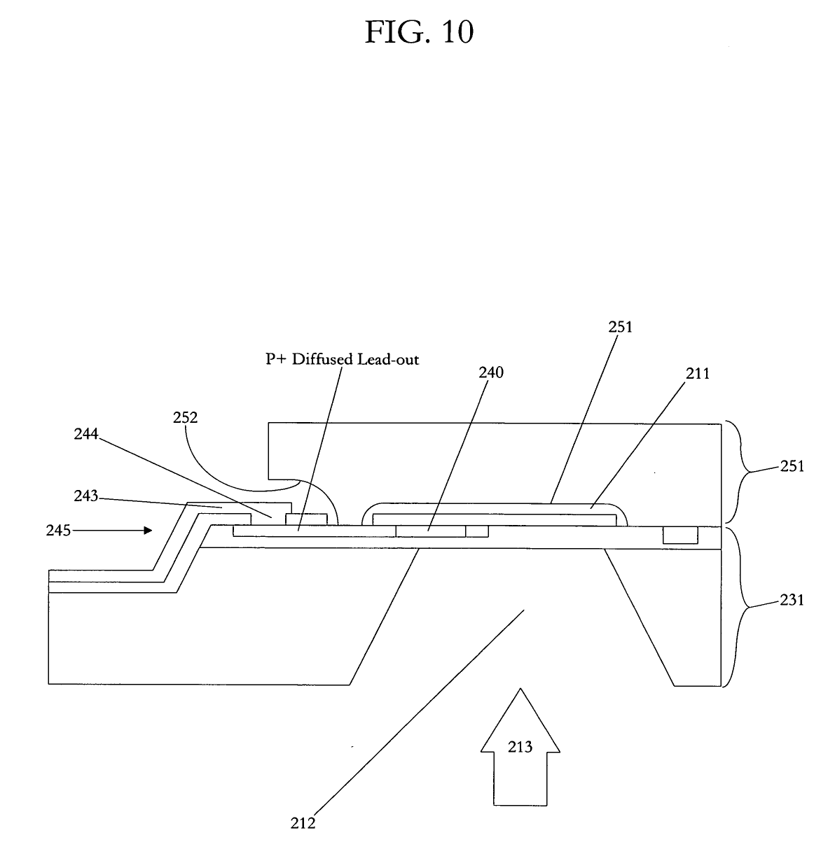

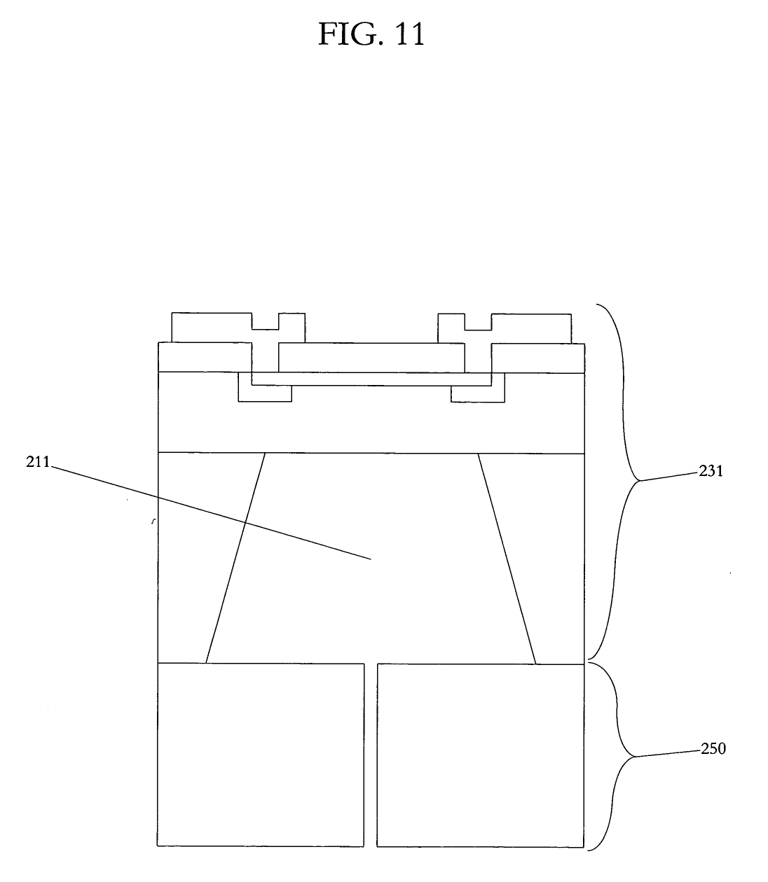

Referring now to FIG. 10, the second layer 250 is a borosilicate glass that is provided with a first depression 251 and a second depression 252. The first depression 251 is dimensioned according to at least one resistive element 240 to provide a cavity 211 that is sealed reference cavity when the second layer 250 is anodically bonded to the first layer 231. In an alternative embodiment, depicted in FIG. 11, the cavity 211 is a reference cavity that is not sealed. Referring again to FIG. 10, the second depression 252 is dimensioned according to the contacts 243, 244 to provide a cover over at least a portion of the conducting material 245, preferably the contacts 243, 244 themselves, when the first layer 231 is anodically bonded to the second layer 250. The depressions 251, 252 are formed by masking the second layer 250 with CrAu and applying an etchant, preferably a buffered oxide etchant, such as HF. Then, the CrAu is stripped off.

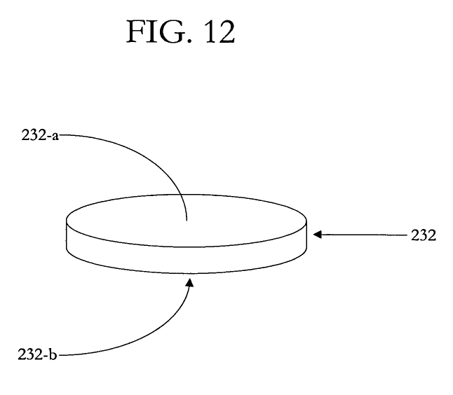

In the embodiment depicted in FIG. 9, the first layer 231 includes pure silicon in a single-crystal structure, preferably P-type silicon. The first layer 231 is fabricated by obtaining a wafer 232, preferably a P-type wafer (shown in FIG. 12), and employing a photolithographic-implant process to create a resistive element 240, preferably a piezoresistive element, within the first layer 231. In the presently preferred embodiment, the first layer 231 is provided with more than one resistive element 240; advantageously, the first layer 231 is provided with a plurality of pairs of resistive elements.

Referring now to FIG. 12, the wafer 232 is provided with a first side 232-a, and, located opposite the first side 232-a, the wafer 232 is provided with a second side 232-b. The wafer 232 is fabricated by first obtaining raw silicon in the form of quartzite. Then, the raw silicon is melted with a carbon, such as coal, coke, or woodchips, in a quartz crucible to form a silicon melt. The silicon melt is composed principally of silicon oxide and silicon carbide. At high temperatures, the silicon oxide and the silicon carbide react chemically to produce pure silicon and gaseous by-products CO and SiO.

The crucible is placed in a high-temperature furnace. Located above the crucible and the silicon melt is a puller which is provided with a seed crystal attached at the tip. The puller is brought down into contact with the silicon melt and then returned to a position outside the silicon melt above the crucible. As the puller is moved above the silicon melt, a continuous deposition of silicon melt adheres to the seed crystal and condenses into a cylinder of single-crystal silicon several feet long with a diameter between 100 and 300 millimeters. The cylinder is ground so that, in cross-section, a perfect circle is formed. Then fine diamond saws are used to slice the cylinder into thin wafers that are P-type wafers.

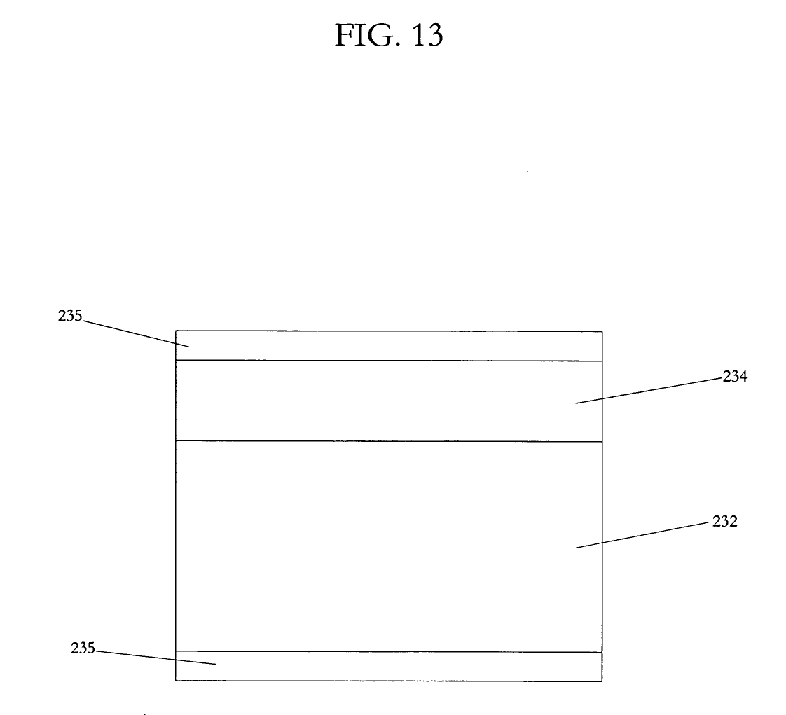

After the cylinder is sliced, additional layers, such as an epitaxial layer 234 and an intermediate layer 235, are formed on the first side 232-a and dopants implanted onto the wafer 232 through a photolithographic-implant process, as is depicted in FIG. 13. In the presently preferred embodiment, an epitaxial N-type layer 234 is formed on the first side 232-a of the wafer 232. After the epitaxial layer 234 is formed, an intermediate layer 235 that is preferably composed of SiO₂ is formed on both the epitaxial layer 234 and the second side 232-b of the wafer 232. Then, the photolithographic-implant process is employed to implant a plurality of dopants into the epitaxial layer 234.

The first step in the photolithographic-implant process involves the forming of a photo-resist layer. In the preferred embodiment, a photo-resist layer is formed on the intermediate layer 235 in a pattern determined by the dopant implant. Then, an etchant is employed to etch through the intermediate layer 235. After the intermediate layer 235 has been etched, the photo-resist layer is removed and the dopant is implanted. In the preferred embodiment, the dopant is implanted through deposition and diffusion. In an alternative embodiment, the dopant is implanted through ion implantation. After the dopant is implanted, the intermediate layer 235 is re-formed. Additional dopants can be implanted by repeating the photolithographic-implant process.

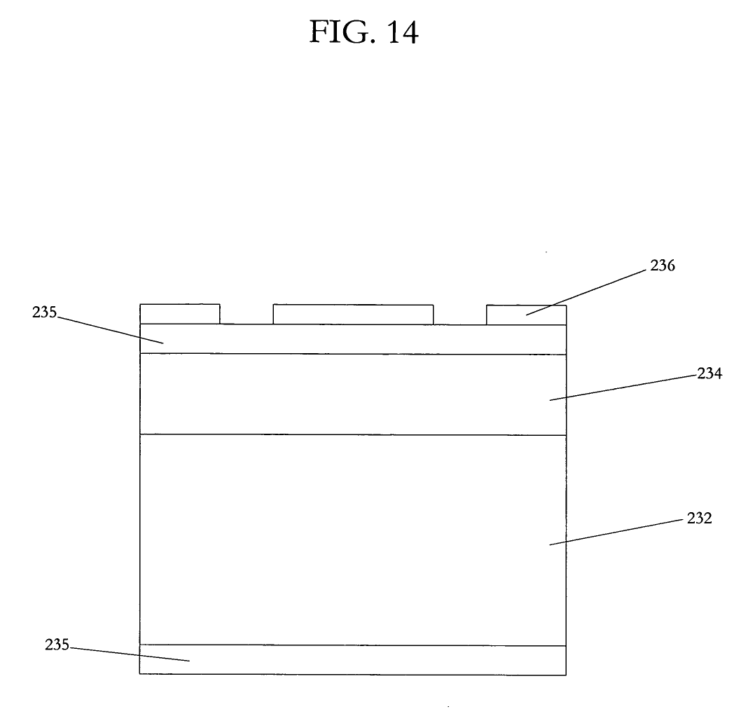

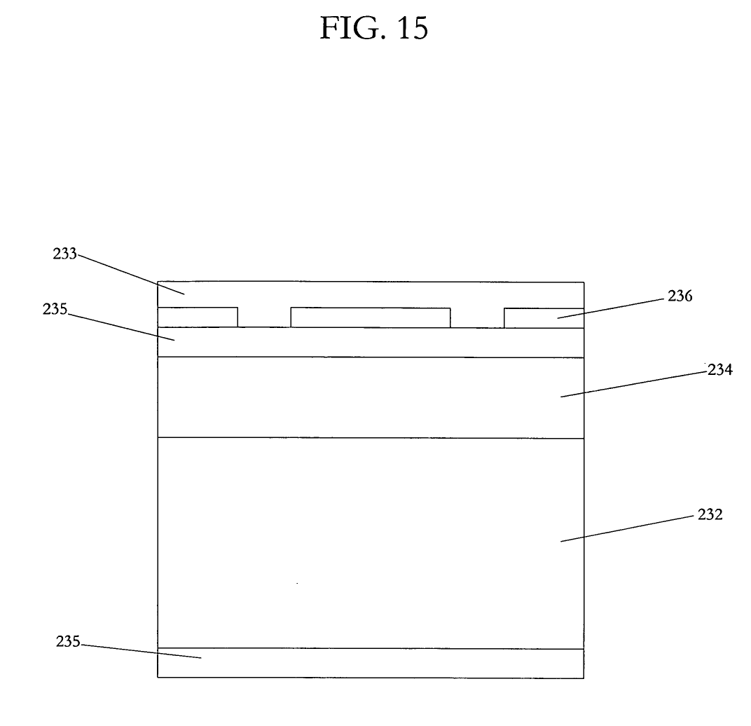

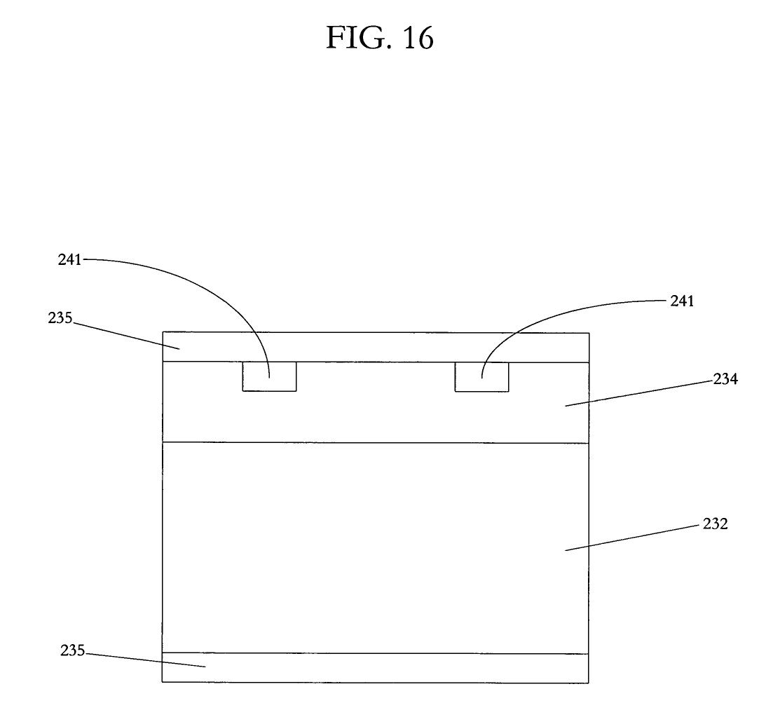

In the preferred embodiment, the resistive element 240, preferably a piezoresistive element, is implanted into the epitaxial layer 234 through the photolithographic-implant process. As depicted in FIG. 14, the resistive element is fabricated by first forming a first photo-resist layer 236 in a pattern determined by a first P+ diffusion. An etchant 233 is employed to etch through the intermediate layer 235, as shown in FIG. 15. Then, the first photo-resist layer 236 is removed and, as shown in FIG. 16, a first P-type material 241, such as boron, is diffused within the epitaxial layer 234. After the first P-type material 241 is diffused within the epitaxial layer 234, the intermediate layer 235 that has been etched is re-formed by regrowing the SiO₂.

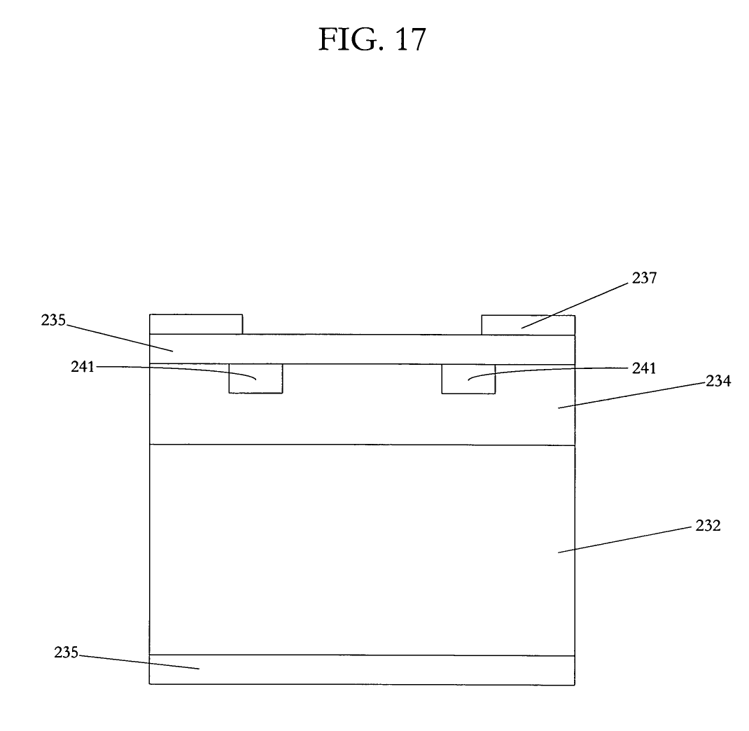

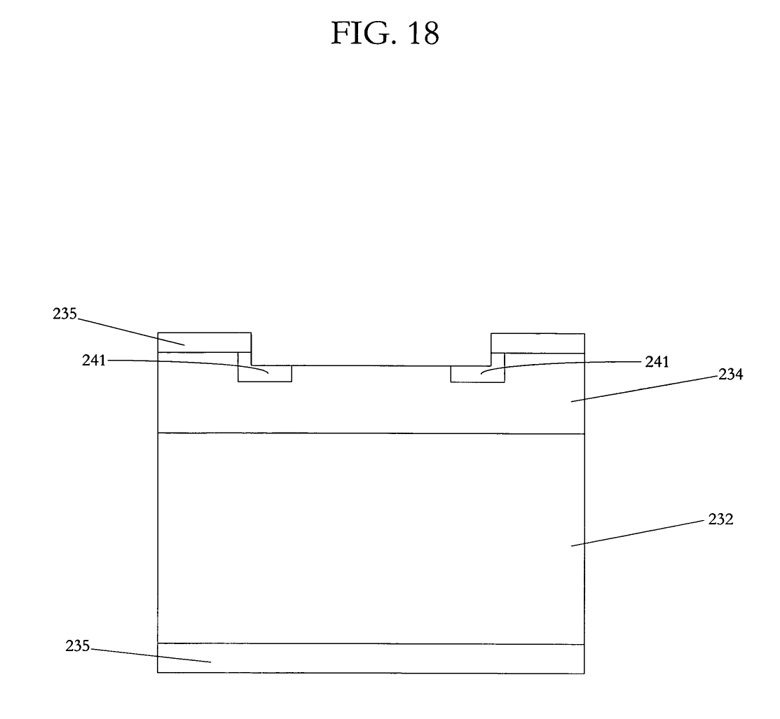

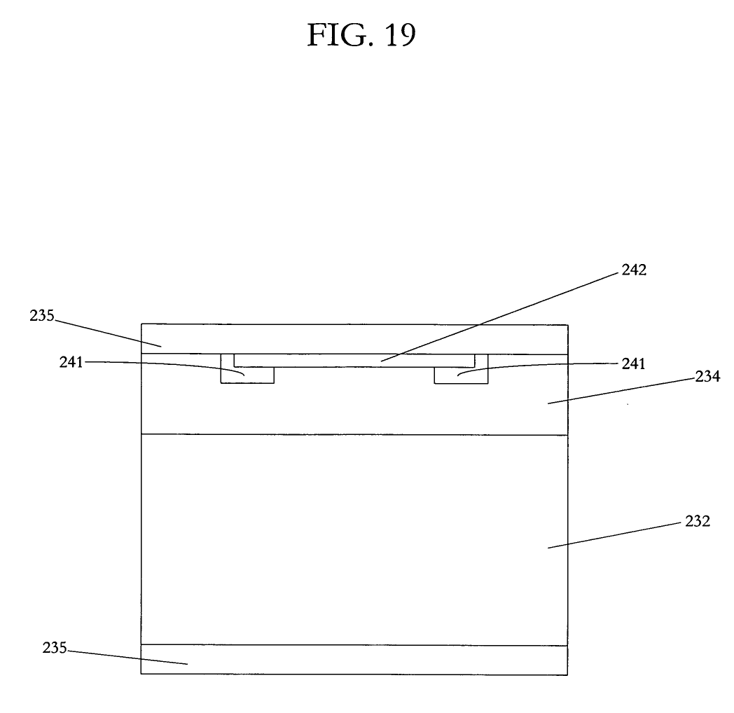

After the intermediate layer 235 is re-formed, as depicted in FIG. 17, a second photo-resist layer 237 is formed on the intermediate layer 235 in a pattern determined by a second P diffusion. An etchant is employed to etch through the intermediate layer 235, as is shown in FIG. 18. Then, the second photo-resist layer 237 is removed and, as depicted in FIG. 19, a second P-type material 242, such as boron, is diffused within the epitaxial layer 234. After the second P-type material 242 is diffused within the epitaxial layer 234, the intermediate layer 235 that has been etched is provided with additional SiO₂.

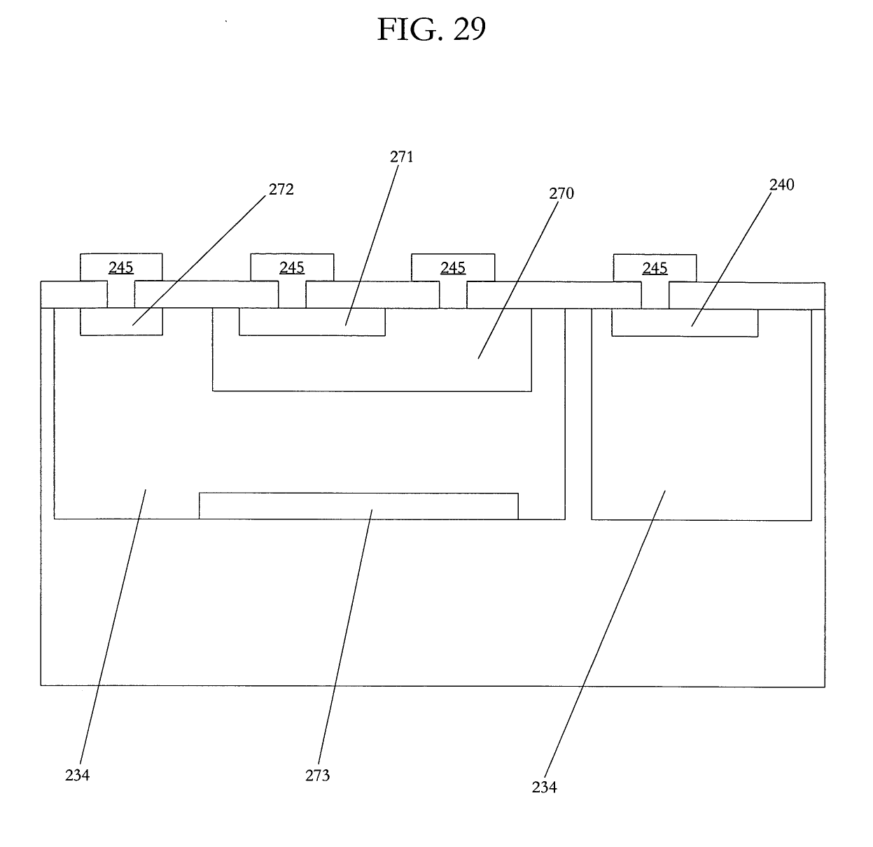

Other dopants are implanted via the photolithographic-implant process. As shown in FIG. 29, a base 270 that is P-type material is implanted in the epitaxial layer 234. Within the base 270, a N-type emitter 271 is implanted via the photolithographic-implant process. Other N-type regions are implanted via the photolithographic-implant process. For example, FIG. 29 depicts a collector 272 that includes N-type material within the epitaxial layer 234. Additionally, FIG. 29 depicts a buried layer 273 that includes N-type material. The buried layer 273 is placed under the collector 272 to reduce resistance and to increase the immunity from latchup.

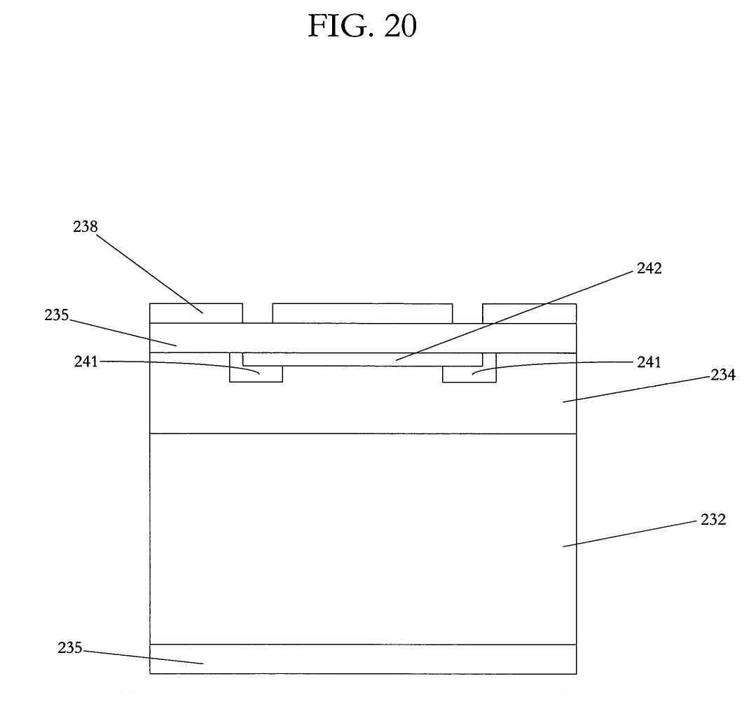

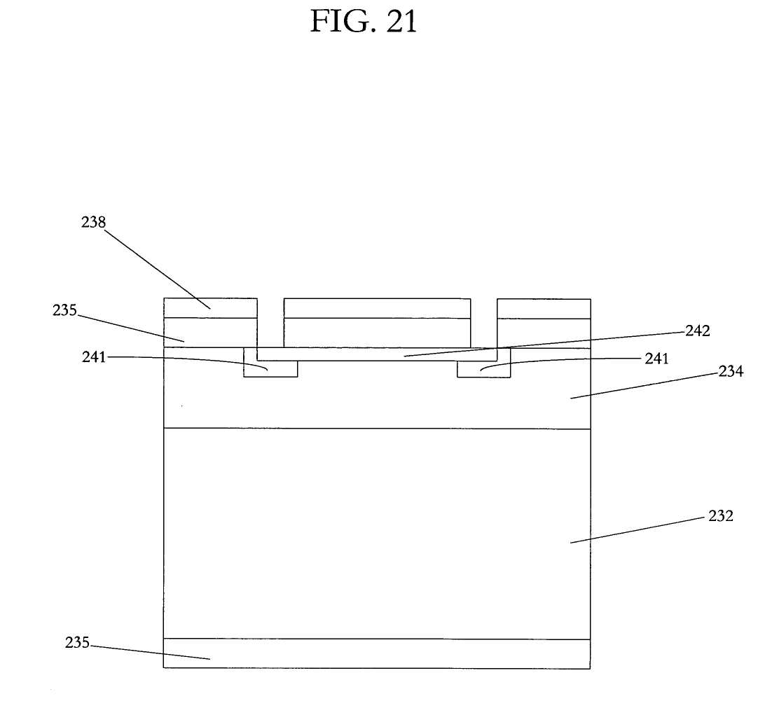

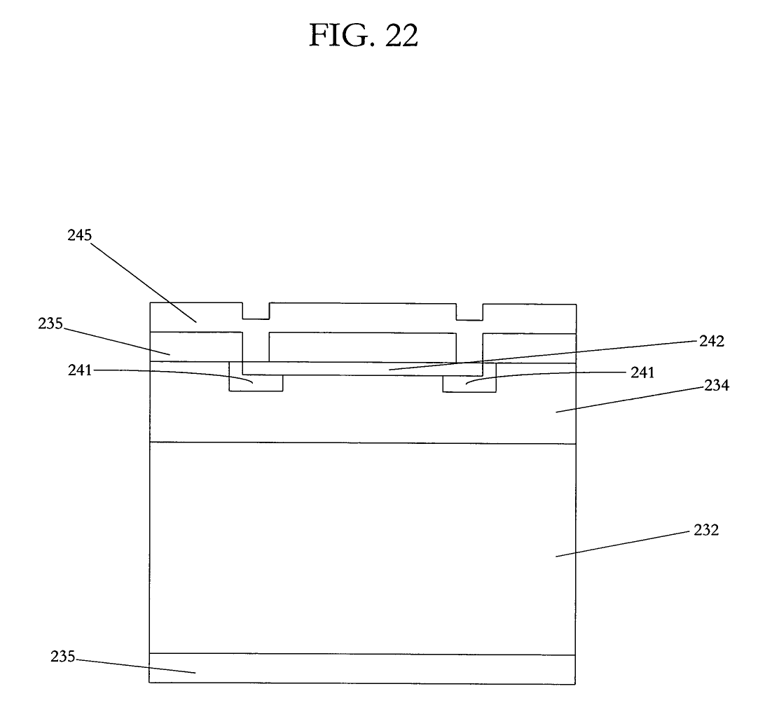

The photolithographic-implant process is employed to implant contacts 243, 244. After the intermediate layer 235 is grown, a third photo-resist layer 238 is formed on the intermediate layer 235 in a pattern determined by a metallization pattern, as is shown in FIG. 20. An etchant is employed to etch through the intermediate layer 235, as shown in FIG. 21. Then, the third photo-resist layer 238 is removed and, as depicted in FIG. 22, a conducting material 245, such as aluminum, is deposited. The conducting material 245 is deposited through electroplating; however, in an alternative embodiment, the conducting material 245 is sputtered and etched/ion milled.

FIG. 22 depicted conducting material 245 that includes aluminum. However, in an alternative embodiment, the conducting material 245 consists of a material that resists electromigration, such as a single layer of gold. A titanium-tungsten (TiW) layer is used under the gold for adhesion to the underlying material.

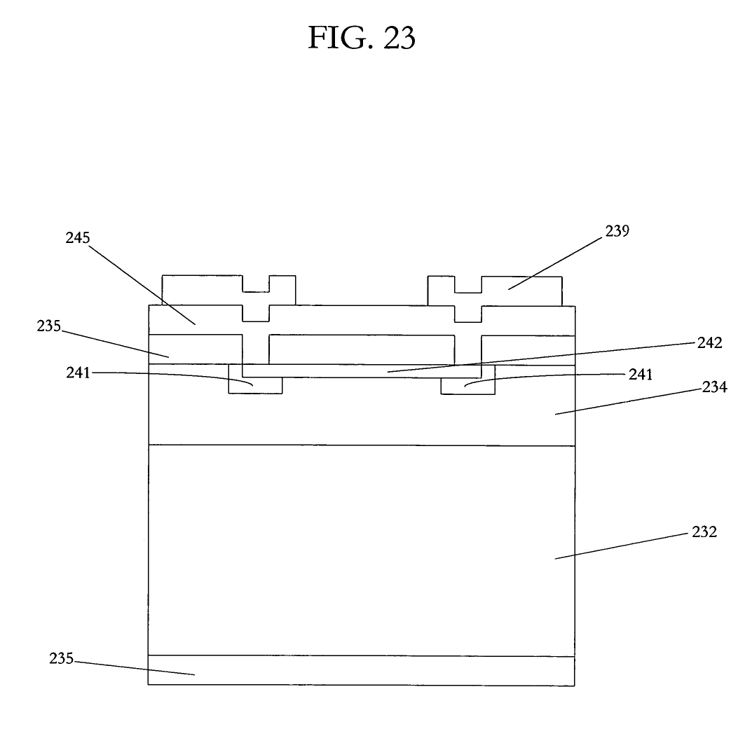

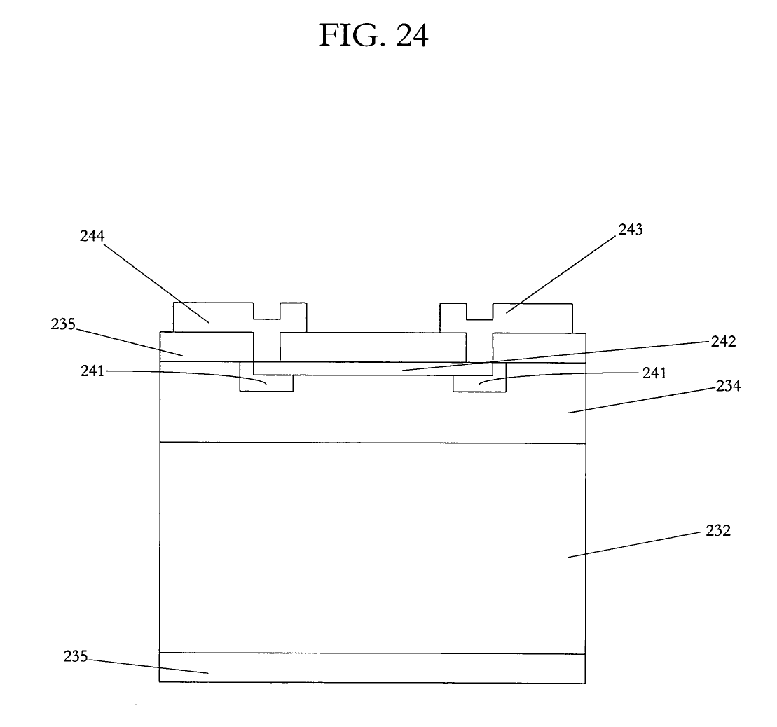

After the conducting material 245 is deposited, a fourth photo-resist layer 239 is formed over the conducting material 245 in a pattern determined by the conducting pattern, as is shown in FIG. 23. An etchant is employed to remove the conducting material 245 that is not covered by the fourth photo-resist layer 239, as depicted in FIG. 24. Then, the fourth photo-resist layer 239 is stripped off.

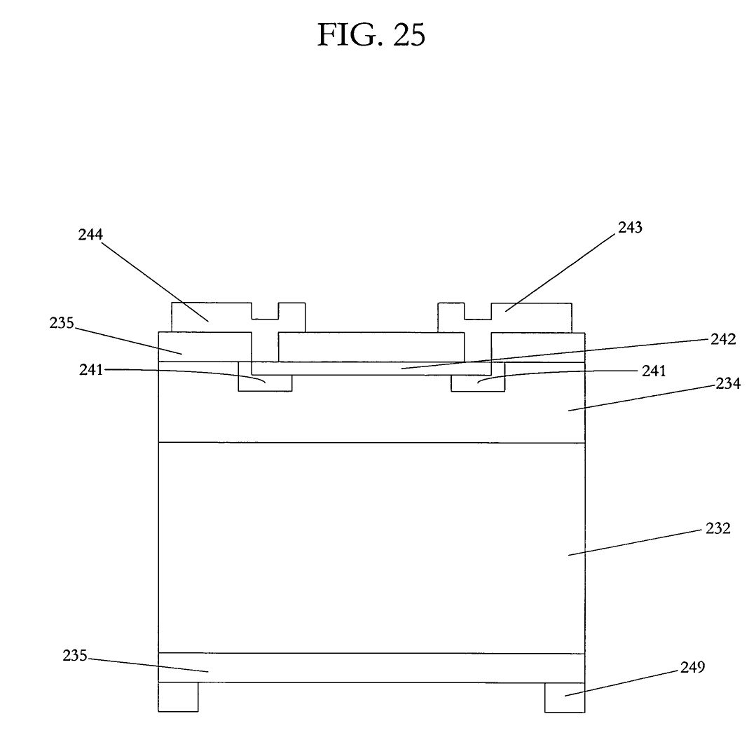

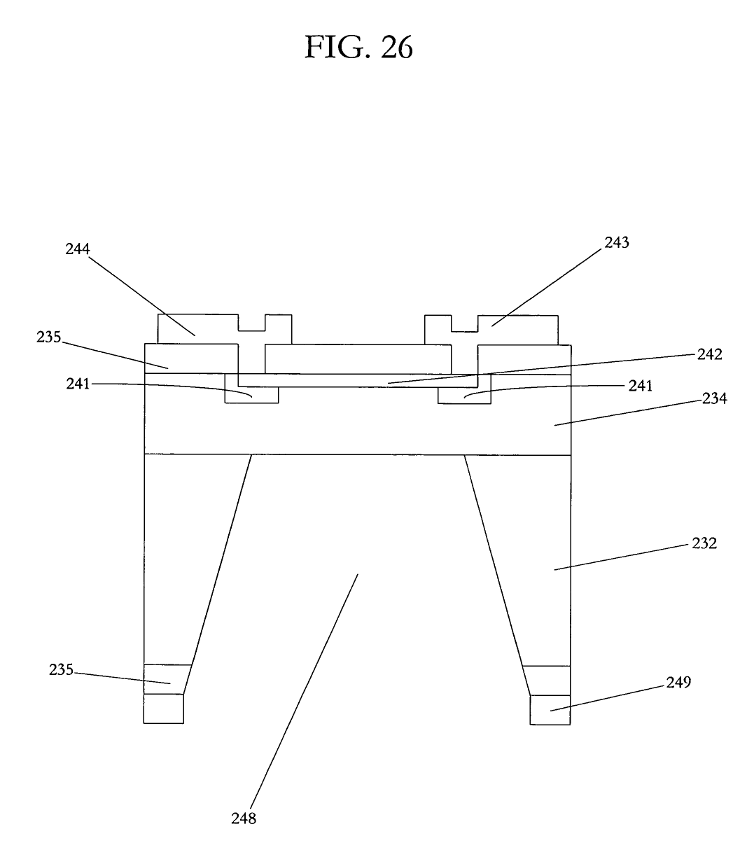

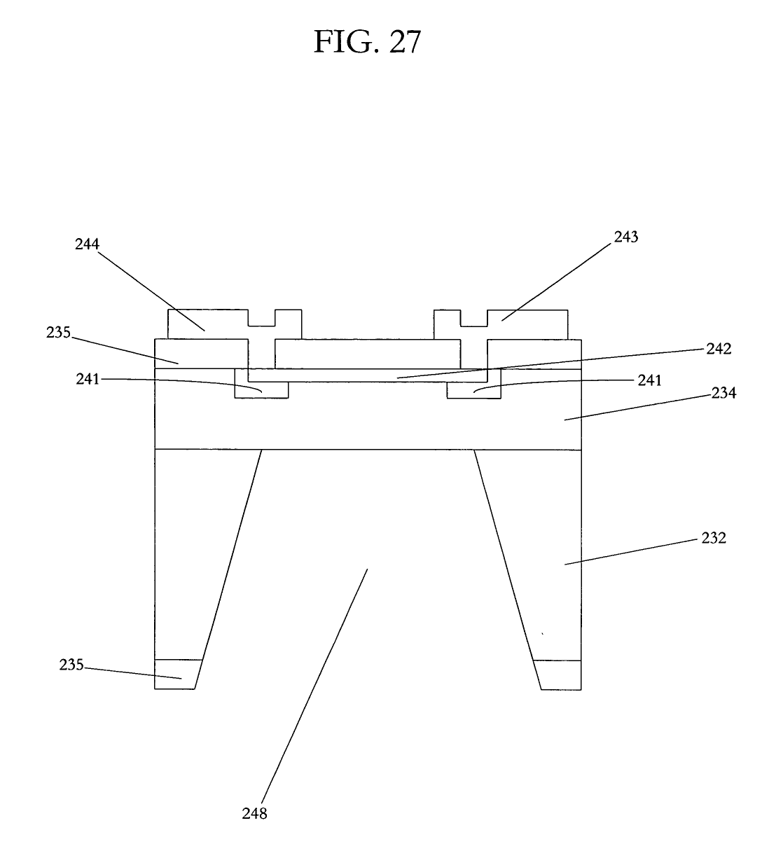

Referring now to FIG. 26, the first layer 231 is provided with a pocket 248 that is formed within the wafer 232. The pocket 248 is dimensioned, at least in part, according to the resistive element 240, and, the preferred embodiment, the pocket 248 is dimensioned according to a plurality of pairs of resistive elements. After the contacts 243, 244 have been implanted, the pocket 248 is formed, as depicted in FIG. 25, by applying a fifth photo-resist layer 249 to the second side 232-b of the wafer 232 in a pattern determined by the dimensions of the pocket 248. The pocket 248 is formed, as is shown in FIG. 26, by applying an etchant, such as KOH, while protecting the conducting material 245 and the intermediate layer 235 located on the first side 232-a of the wafer 232. After the pocket 248 is formed, the fifth photo-resist layer 249 is removed, as depicted in FIG. 27.

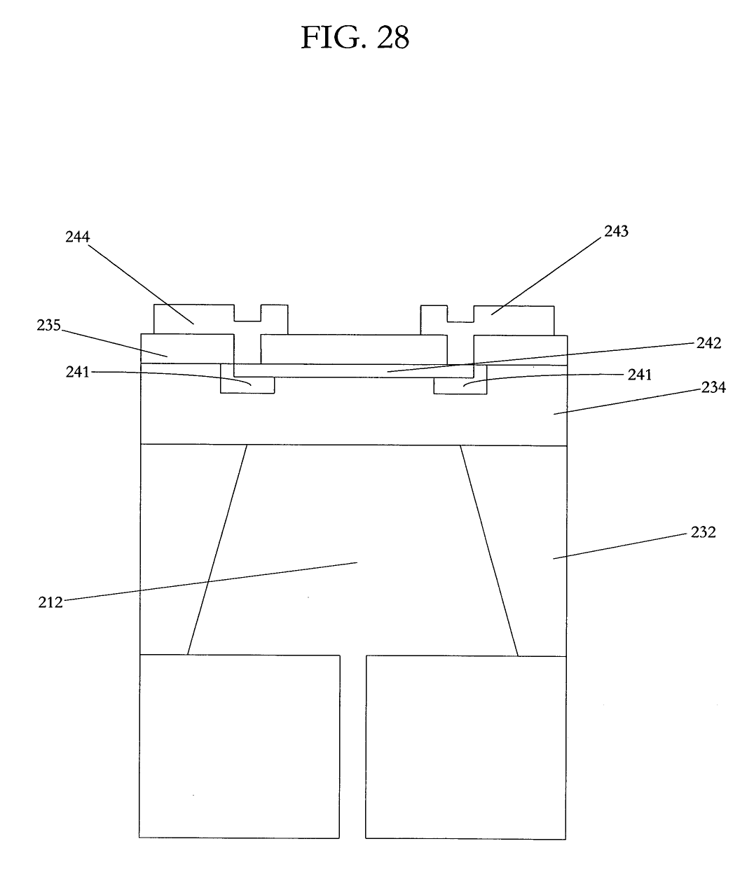

As shown in FIG. 10, the pocket 248 is shaped to form a cavity 212. In the preferred embodiment, the cavity 212 is an input cavity that is configured to receive an external stimulus 213, such as pressure. In an alternative embodiment, the cavity 212 is a sealed reference cavity when the second side 232-b of the wafer 232 is anodically bonded to the second layer 250, as is shown in FIG. 9. In yet another alternative embodiment, depicted in FIG. 28, the cavity 212 is a reference cavity that is not sealed.

After the pocket 248 is formed, the first layer 231 and the second layer 250 are bonded together. In the preferred embodiment, the first layer 231 is anodically bonded to the second layer 250. First, the first and second layers 231, 250 are heated to a temperature in the range of 300 to 500° C. to cause the alkali-metal ions in the first layer 231 to become mobile. The first and second layers 231, 250 are brought into contact and a high voltage applied across them to cause the alkali cations to migrate from the interface and oxygen anions from the first layer 231 to the second layer 250.

After the first and second layers 231, 250 are bonded, it is preferable that the first layer 231 be ground and polished. Then, advantageously, the first and second layers 231, 250 are thinned via HF. After the first and second layers are thinned and bonded, the sensing element 230 is bonded within the housing 220, preferably with an RTV rubber, such as a fluoro-silicon RTV rubber. Advantageously, RTV 730 manufactured by Dow Corning is used to bond the sensing element 230 within the housing 220.

Referring now to FIG. 30, the conducting material 245 provides a lead 260 that is connected to the processing module 300. Via at least one lead 260, the sensing component 210 sends a signal 240 that is electrical in nature to the processing module 300. According to one aspect, the signal 240 is a voltage. According to another aspect, the signal 240 is an electrical current. Advantageously, the magnitude of the signal 240 is determined according to the external stimulus 213, which, in the case of the presently preferred embodiment, is pressure.

Referring now to FIG. 4, the processing module 300 includes an operational amplifier 310 that is provided with a low pass filter 311, depicted as a capacitor 312 in parallel with a resistor 313. The operational amplifier 310 amplifies the signal 240 from the sensing component 210, and the low pass filter 311 filters out unwanted frequencies and noise. In the embodiment shown in FIG. 4, the microcontroller 330 is provided with an analog-to-digital converter 320 and memory 340. However, in an alternative embodiment, the medical device 100 is provided with an analog-to-digital converter 320 and memory 340 that are separate from the microcontroller 330.

After the signal 240 is digitized in the analog-to-digital converter 320, the signal 240 is stored in memory 340. Advantageously, the signal 240 is stored in memory 340 as a function of time as data 321. As shown in FIG. 4, the microcontroller 330 is provided with a processing unit 331 that is capable of performing mathematical operations on the data 321, such as detecting changes in the magnitude of the stimulus 213 and the rate of any change in the magnitude of the stimulus 213.

After the signal 240 is stored in memory 340, a communications module 400 is preferably employed to link the memory 340 to a computer 700. According to one aspect, the communications module 400 is a low power RF transceiver 414 integrated with the microcontroller 330. According to one aspect, the communications module 400 is a wireless module 410, such as an infared transmitter. According to another aspect, the communications module 400 is a USB controller 420 and a USB cable 421. According to yet another aspect, the communications module 400 is a display 430, such as an LCD display.

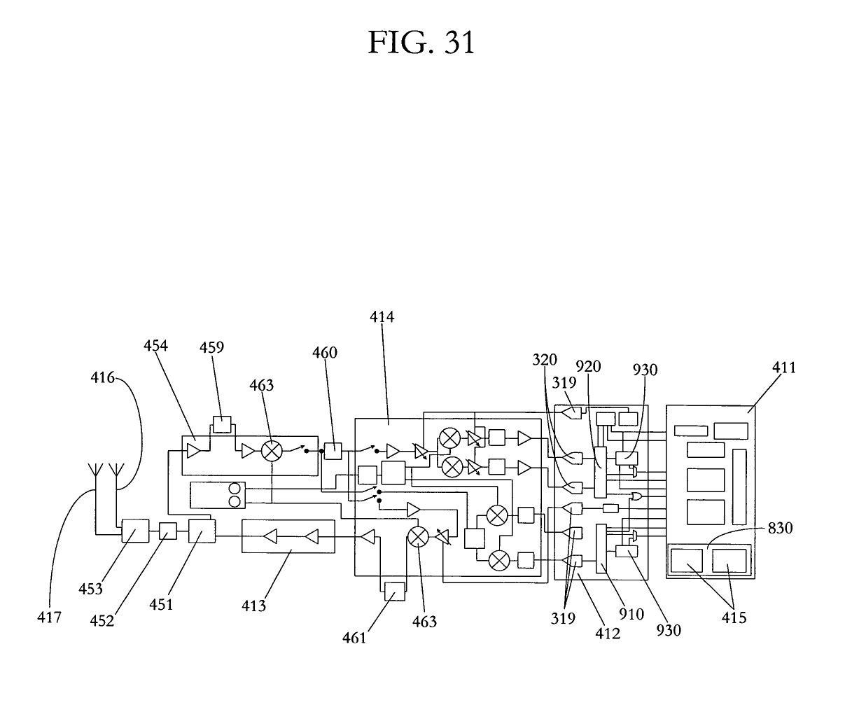

In the preferred embodiment, shown in FIG. 31, the wireless module 410 includes a media access controller 411, a baseband controller 412, a power amplifier 413 (preferably a linear power amplifier), a transceiver 414, such as an RF/IF transceiver, memory 415, a first antenna 416, a second antenna 417, a synthesizer 450, a transmission-receiving switch 451, an RF bandpass filter 452, an antenna switch 453, and an LNA mixer 454. As shown, the wireless module is provide with a plurality of filters 459, 460, 461.

Advantageously, the synthesizer 450 and the RF/IF transceiver 414 are integrated into an integrated controller 480. Consequently, in another alternative embodiment, the wireless module includes an integrated controller 480. In yet another alternative embodiment, the RF/IF transceiver 414 and the microcontroller 330 are integrated into a transceiver control unit 419.

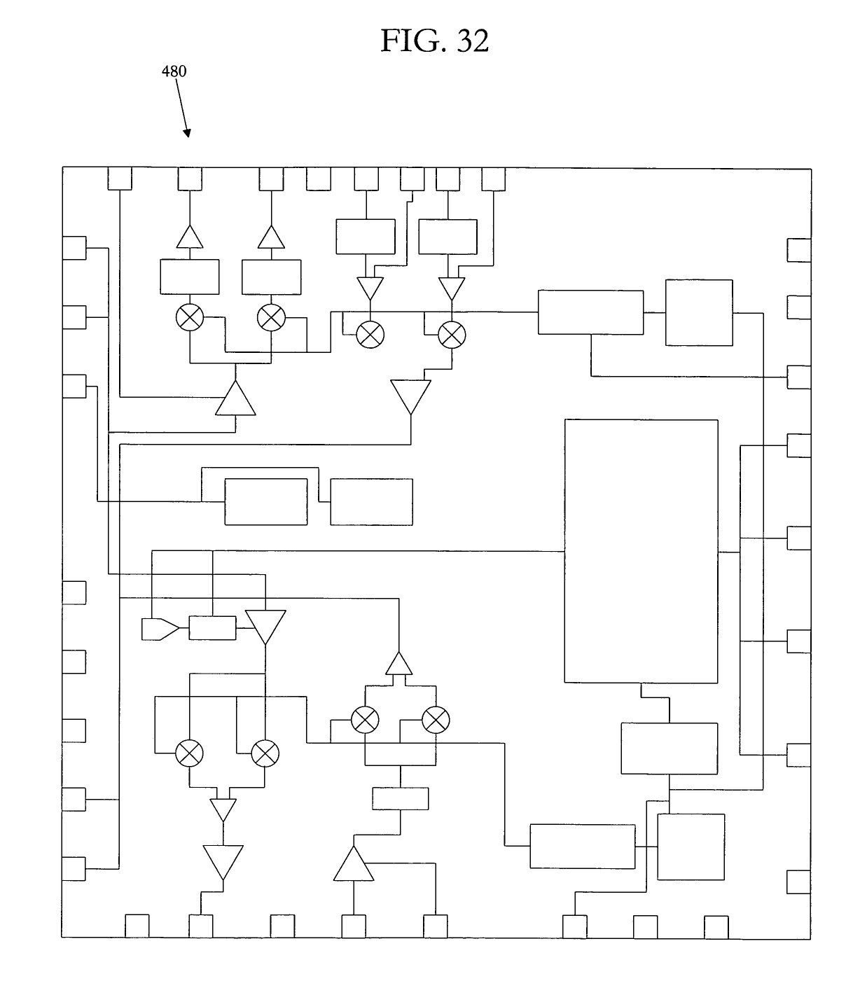

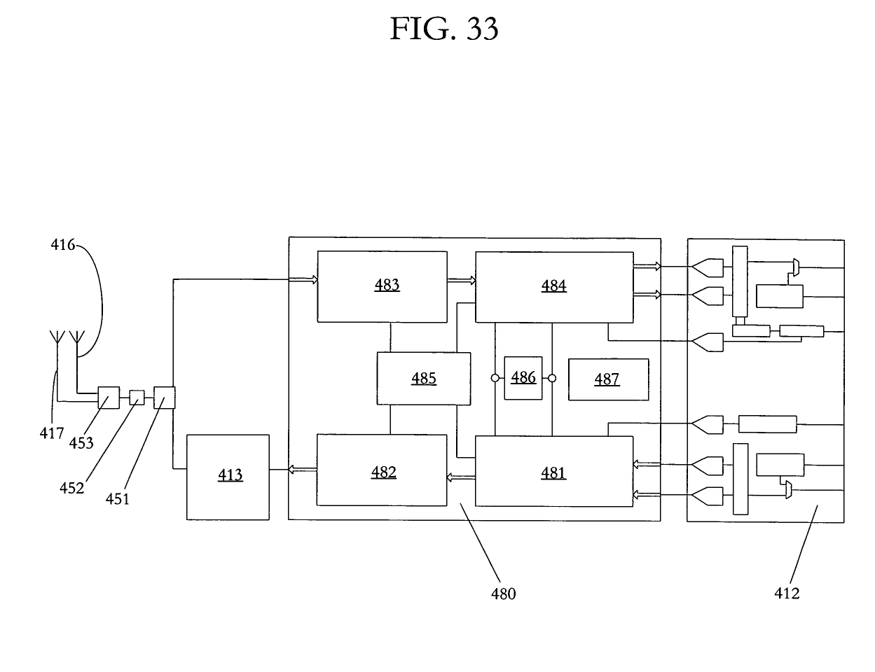

The integrated controller 480 is shown in FIG. 32 and diagrammatically in FIG. 33. As depicted in FIG. 33, the integrated controller 480 is provided with an intermediate frequency transmission stage 481 and a signal transmission stage 482. The integrated controller 480 is also provided with a signal receiving stage 483 and an intermediate frequency receiving stage 484. Additionally, the integrated controller 480 is provided with an RF-IF synthesizer 485 (which includes a voltage controlled oscillator) and a SAW filter 486. Finally, as FIG. 33 depicts, the integrated controller 480 is provided with an SPI control interface 487.

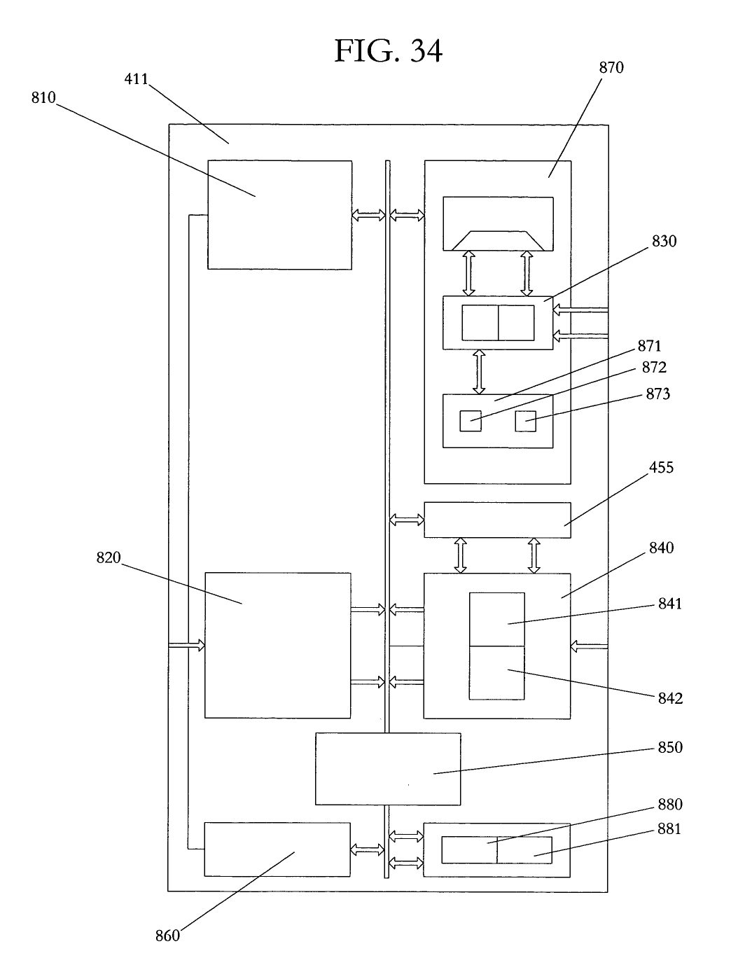

FIG. 34 depicts the media access controller 411 in greater detail. As shown therein, the media access controller 411 is provided with a microcontroller 810, a bus controller 820, such as a USB controller, a memory interface 830, a data encryption module 455 that encrypts and decrypts data, and an attachment interface 840 that includes transmission and reception FIFOs. The media access controller 411 also includes a decoder/arbiter/bridge 850 that manages bus traffic. Additionally, the media access controller 411 includes an interrupt controller 860, a memory controller 870 that manages internal and external memory, and a plurality of timers 880, 881.

The microcontroller 810 is provided with an arithmetic logic unit (“ALU”) that accommodates 32 bits and a plurality of 32 bit registers. The memory controller 870 is provided with internal memory 871 that includes ROM 872 and SRAM memory 873 as well as internal and external memory interfaces. Advantageously, the wireless module 410 includes external flash memory and external SRAM memory.

Referring again to FIG. 31, the wireless module 410 is provided with a baseband controller 412. The baseband controller 412 is provided with a plurality of digital-to-analog converters 319 as well as a plurality of analog-to-digital converters 320. The baseband controller 412 includes a modulator 910 and a demodulator 920, as well as a header 930.

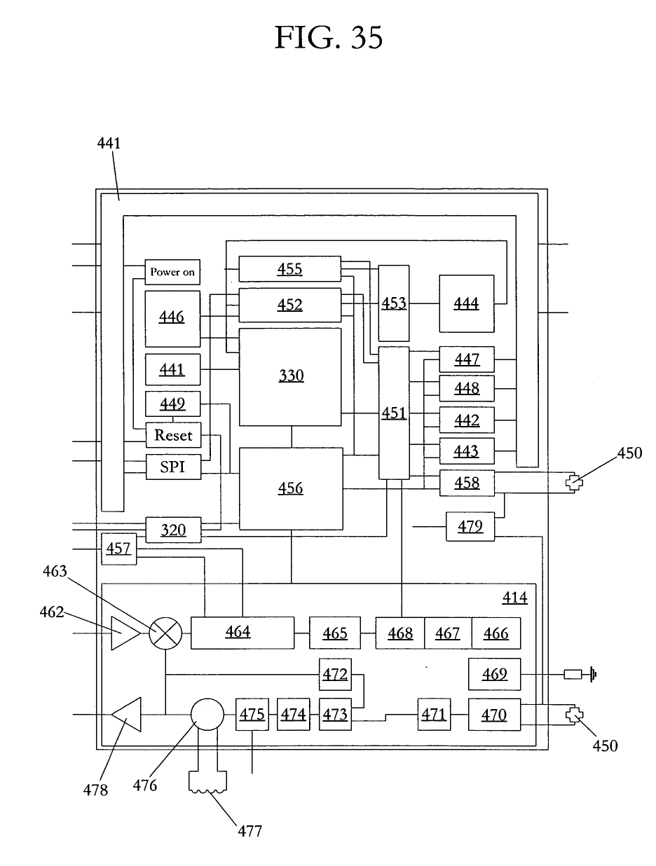

FIG. 35 depicts the transceiver control unit 419. As shown therein, the transceiver control unit 419 is provided with a programmable I/O 441, a general purpose I/O 442, and a UART 443. The transceiver control unit 419 includes a 128 byte SRAM module 444, a 2048 SRAM module 445, a 32 kB flash memory module 446, a flash programming DMA 452, and a RAM arbiter 453. The transceiver control unit 419 is also provided with timers 447, 448, 449, a real time clock 458 that is connected to a crystal 450 and a clock multiplexer 479. As depicted in FIG. 35, the transceiver control unit 419 includes a microcontroller 330 and special function registers 456 as well as an interrupt controller 451. Advantageously, the transceiver control unit 419 includes a data encryption module 455 that encrypts and decrypts data. FIG. 35 also depicts the transceiver control unit 419 with an analog-to-digital converter 320 and a multiplexer 457.

The transceiver control unit 419 is shown including the RF/IF transceiver 414. As shown therein, the RF/IF transceiver 414 is provided with a low noise amplifier 462 that is connected to a mixer 463 that converts an RF signal down to an intermediate frequency. The mixer 463 is connected to a signal module 464 that amplifies and filters the intermediate frequency signal. The signal module 464 is, in turn, connected to a modem 465. The transceiver 414 is provide with an RF buffer 466, a register encoder 467, and a plurality of control registers, referred to collectively as 468. The transceiver 414 is also provided with a bias 469 and a bias resistor. A crystal 450 is connected to a main crystal oscillator 470 which, in turn, is connected to one of the frequency dividers 471, 472. Further, the transceiver 414 is provided with a phase detector 473, a charge pump 474, an internal loop filter 475, and a coltage controlled oscillator 476 and a VCO inductor 477, as well as a power amplifier 478.

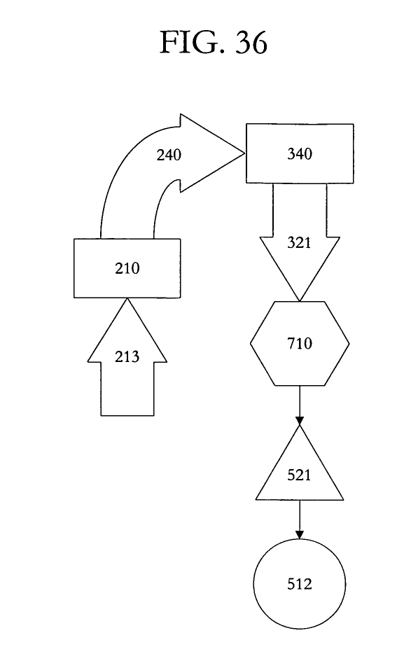

Referring now to FIG. 36, after the computer 700 is linked to the medical device 100 via the communications module 400, the data 321 is transmitted to the computer 700, preferably to a port 710 on the computer 700. According to one aspect, the port 710 is a wireless module 410 connected to the computer 700. According to another aspect, the port 710 is a USB port. According to another aspect, the port 710 is a serial port or a parallel port. According to yet another aspect, the port 710 is an infared receiving port. According to yet another aspect, the port 710 receives an Ethernet cable or a telephone line.

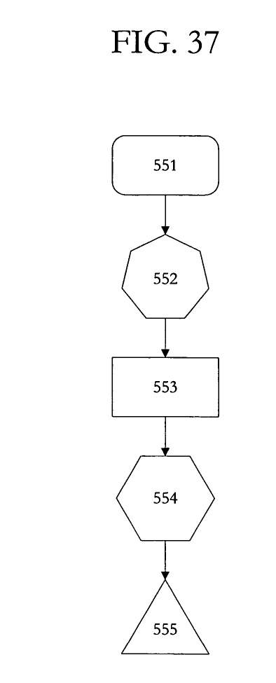

The computer 700 obtains the data 321 by running an acquisition routine 550, preferably within the port 710. Alternatively, the acquisition routine 550 is run within a software routine 510. The acquisition routine 550 is depicted FIG. 37. As shown in step 551, the acquisition routine 550 transmits a code that is unique to the sensing module 200 and prompts the sensing module 200 to begin transmitting data 321. Referring now to step 552, the communications module 400 within the sensing module 200 transmits a confirmation code followed by the data 321. The acquisition routine 550 then obtains the confirmation code and the data 321, as depicted in step 553. Advantageously, as shown in step 554, the confirmation code is checked to ensure that the proper sensor is transmitting. After the confirmation code is checked and verified, the acquisition routine 550 obtains the data 321, as is depicted in step 555.

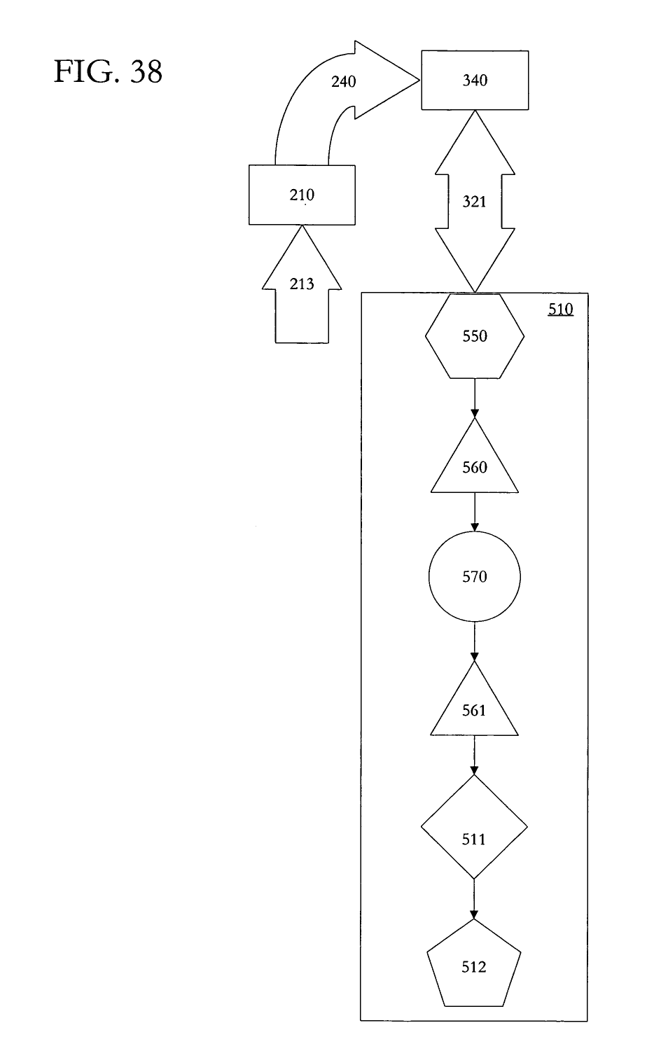

Referring now to FIG. 38, after the software routine 510 obtains the data 321 from the port 710, the software routine 510 stores the data 321 into a database 521 located in memory within the computer 700 via a store operation, as depicted in step 560. According to one aspect, the computer 700 is a local computer 720. According to another aspect, the computer 700 is a server 530. Advantageously, the data 321 is stored into a database 521 that is located in memory within both the local computer 720 and on the server 530. In the preferred embodiment, the database 521 is networked so that access to the database 521 is provided via the internet. Within the software routine 510, a graphing subroutine 570 graphs the data 321 as a function of time. Preferably, the graphing subroutine 570 graphs the data 321 so that it can be read by an internet browser 516, such as Internet Explorer®. As shown in FIG. 38, the graphed data is stored into the database 521 via a second store operation 561.

After the data 321 is stored, the software routine 510 performs a data analysis routine 511, as depicted in step 580. According to one aspect, the data analysis routine 511 determines whether the stimulus 213 has reached or dropped to a predetermined level. According to another aspect, the data analysis routine 511 determines whether the rate of change in the magnitude of the stimulus 213 has attained a predetermined rate.

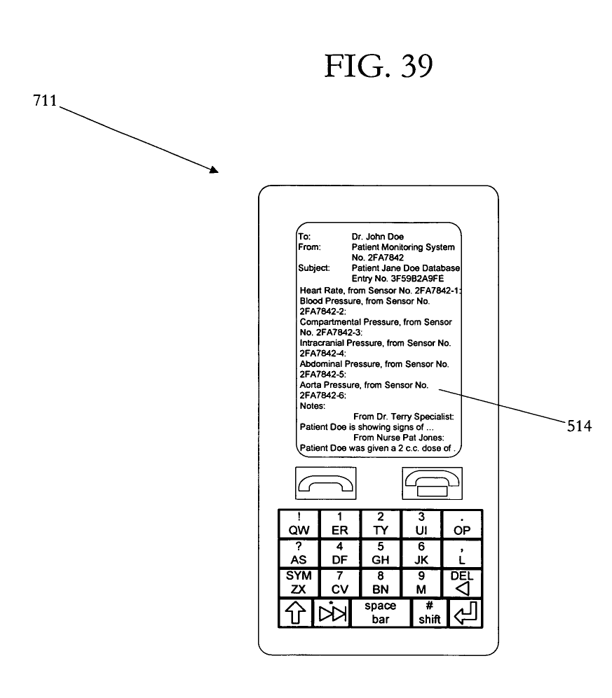

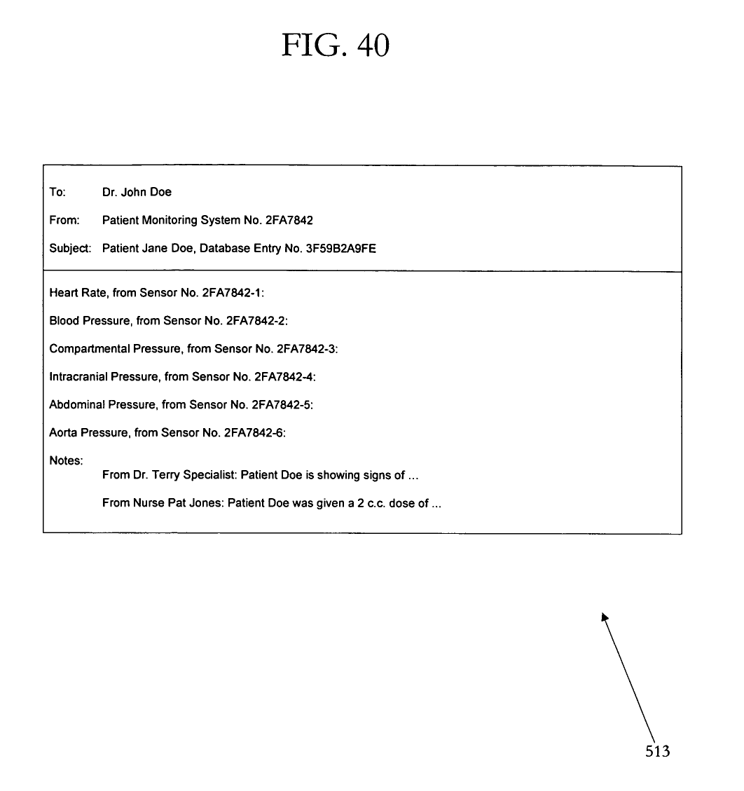

If the data analysis routine 511 determines that the stimulus 213 has reached or dropped to a predetermined level, or that the rate of change in the magnitude of the stimulus 213 has attained a predetermined rate, an alert 512 is transmitted, preferably to a handheld communications device 711, as depicted in FIG. 39. According to one aspect, the alert is an e-mail 513, as shown in FIG. 40. According to another aspect, the alert 512 is a text message 514. According to yet another aspect, the alert 512 is a page. Advantageously, the alert 512 includes relevant data 321, such as the magnitude of the stimulus 213, the rate of change in the stimulus 213, a graph of the data from the graphing module, or a URL or other link to where the data 321 is located in the database 521.

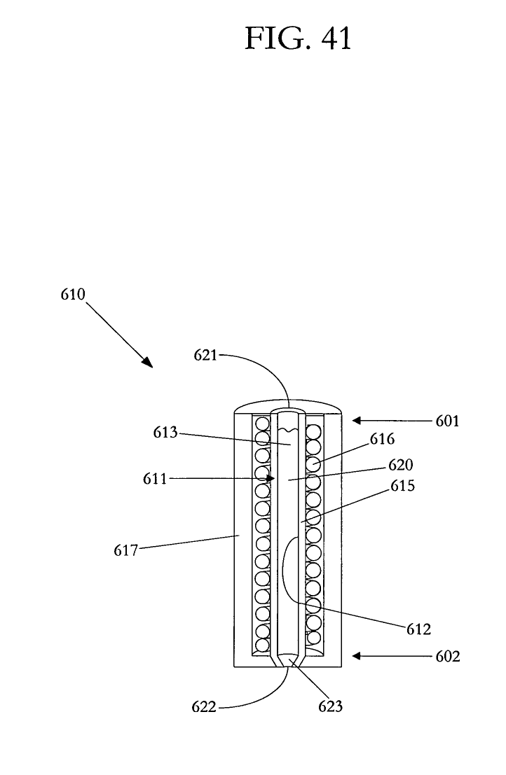

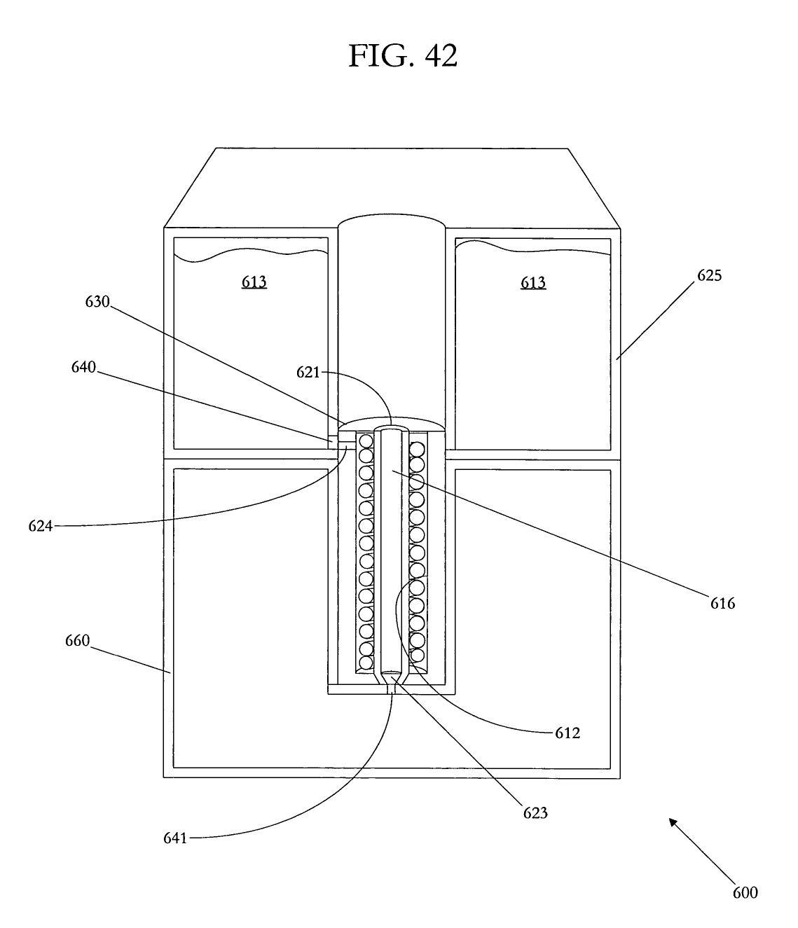

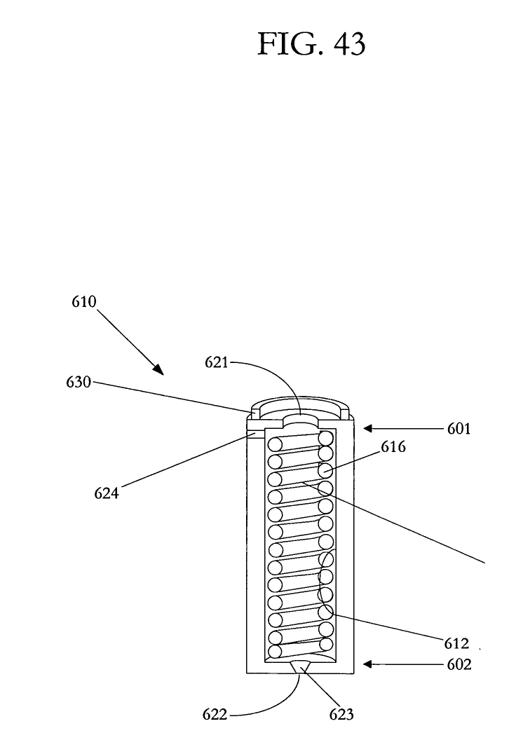

Referring now to FIG. 41 and FIG. 42, the presently preferred embodiment is provided with a retaining device 600 that includes a sterilizer 610 with a first end 601 and a second end 602. Advantageously, as shown in FIG. 43, the retaining device 600 is also provided with an electrical charger 630 that re-charges the power supply of the medical device 100, such as, by re-charging a battery located within the medical device 100. As shown in FIG. 43, the electrical charger 630 is located at the first end 601.

FIG. 41 depicts the sterilizer 610 in cross section. As shown therein, the sterilizer 610 is provided with a sensor acceptor 611. The sensor acceptor 611 is shaped according to the sensing component 210, preferably the housing 220. In the preferred embodiment, the sensor acceptor 611 is generally cylindrical in shape.

The sensor acceptor 611 is shown in FIG. 41 containing a fluid 613. According to one aspect, the fluid 613 is water. According to another aspect, the fluid 613 is a solution, such as a sterilizing solution. According to another aspect, the fluid 613 is a saline solution. According to yet another aspect, the fluid 613 is a solvent.

The sensor acceptor 611 includes a heat conducting layer 615 that is fabricated from a material that conducts heat and that holds the fluid 613 when the fluid is heated to at least 220° F. In the preferred embodiment, the sensor acceptor 611 is fabricated from aluminum; however, in an alternative embodiment, the sensor acceptor is fabricated from copper. As shown in FIG. 41, the heat conducting layer 614 is provided with a wall 612 that defines a sensor cavity 620. The sensor cavity 620 is shaped according to the housing 220, such as a generally cylindrical shape. Located adjacent to the heat conducting layer 615 is at least one heating element 616. Preferably, the heating element 616 is an electrical heating element that substantially surrounds the heat conducting layer 615. Located adjacent to the heating element 616 is an insulating layer 617 that is fabricated from a material that resists the conduction of heat, such as a urethane or a polymer including glass fibers.

FIG. 43 depicts an alternative embodiment wherein the heating element 615 is located within the sensor cavity 620. In such an embodiment, the insulating layer is provided with a wall 612 that defines a sensor cavity 620 and is shaped according to the housing 220. The heating element 616 is located adjacent to the wall 612 and preferably surrounds the sensor cavity 620.

The sensor cavity 620 is provided with a first opening 621 and a second opening 622.

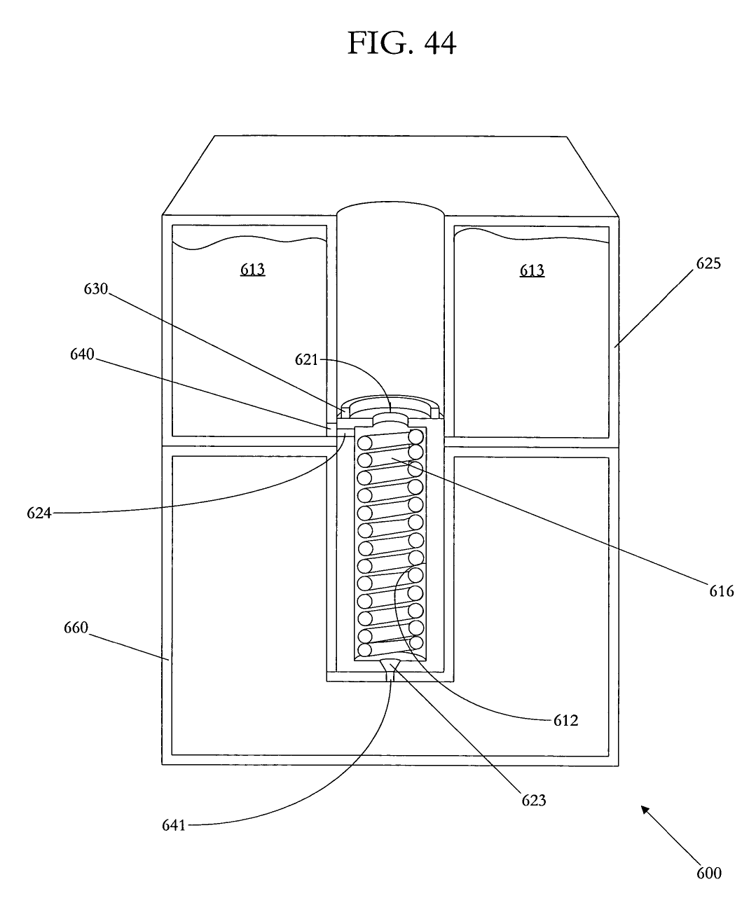

FIG. 43 depicts the first opening 621. As shown therein, the first opening 621 is shaped to provide an insertion clearance between the wall 612 and the housing 220. The second opening 622 is shaped to provide a drain 623 for the fluid 613. Located at the first end 601 is a fluid duct 624 that fluidly connects the sensor cavity 620 to a fluid reservoir 625 where fluid 613 is stored. As shown in FIG. 42 and FIG. 44, a valve 641 is located at the second end 602 of the sterilizer 61 that controls drainage of the fluid 613 into a drainage compartment 660.

In operation, the sensor cavity 620 is filled with fluid 613. Advantageously, the fluid 613 is provided via the fluid duct 624 from the fluid reservoir 625 shown cross-sectionally in FIG. 42 and FIG. 44. The fluid duct 624 is provided with valves 640, 641 that are controlled via the microcontroller 330 of the medical device 100. In an alternative embodiment, however, the retaining device 600 is provided with its own microcontroller. An input from the retaining device 600 is fed to the microcontroller 330. Based upon the input, the microcontroller 330 opens or closes the valves 640, 641.

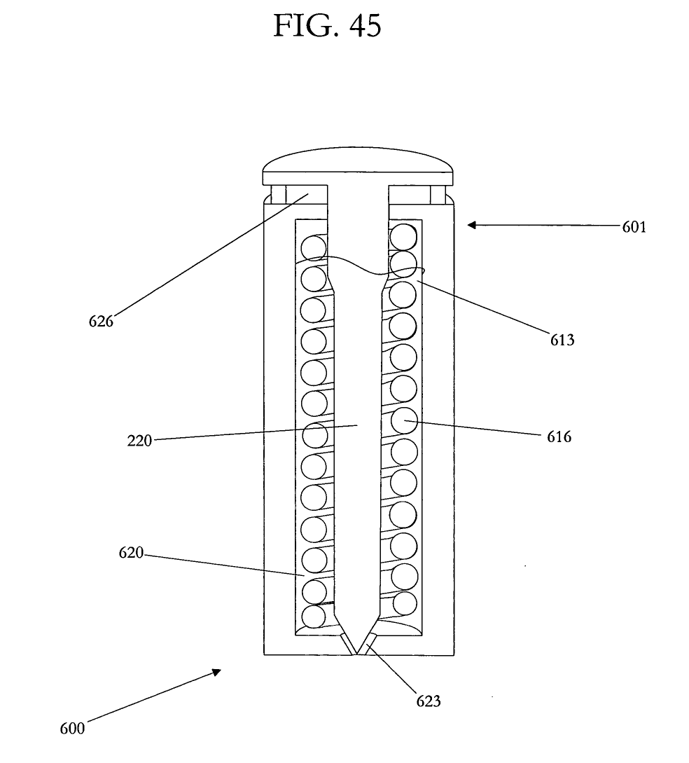

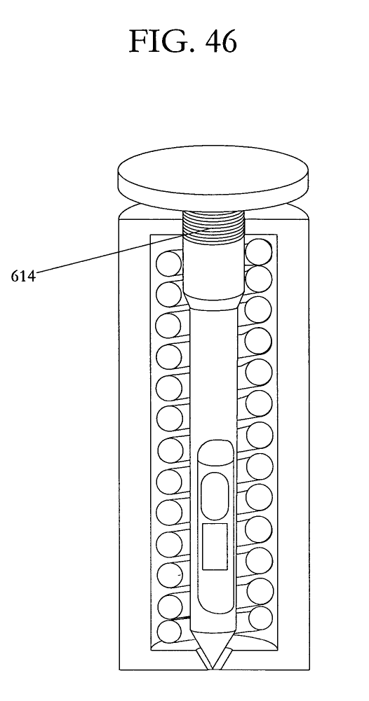

Advantageously, the housing 220 is fluidly sealed within the sensor cavity 620 via a sealing ring 626 located at the first end 601, as shown in FIG. 45. In the preferred embodiment, the sealing ring 626 is fabricated from a rubber or a polymer. As shown in FIG. 46, the sealing ring 626 is configured to be compressed to provide a fluid-tight seal, such as through engagement of threads 614 at the first opening 621. After the housing 220 is sealed within the sensor cavity 620, the heating element 616 heats the fluid 613 to at least 220° F. for at least two minutes. After the fluid is heated to at least 220° F. for at least two minutes, the drain 623 is opened and the fluid 613 is drained from the sensor cavity 620. After the fluid 613 is drained, the sensor cavity 620 is flushed with fresh fluid 613 from the fluid reservoir 625 via the fluid duct 624.

While this invention has been particularly shown and described with references to preferred embodiments thereof, it will be understood by those skilled in the art that various changes in form and details may be made therein without department from the spirit and scope of the invention as defined by the appended claims.

What is claimed is:

1. A medical device comprising:
a) a sensing module including a sensing component provided with a housing that includes an end that is shaped to pierce through a facial layer;
b) the housing includes a diameter that is less than 1.2 millimeters and encloses a sensing element
c) the sensing element includes a first layer and a second layer that are bonded together to form a cavity;
d) the first layer includes a resistive element that is electrically connected to a processing module;
e) the processing module includes an analog-to-digital converter that receives a signal from the resistive element and that is electrically connected to a processing unit; and
f) the processing module is electrically connected to a communications module.

2. A medical device according to claim 1, wherein the resistive element is a piezoresistive element

3. A medical device according to claim 1, wherein the communications module is a wireless module.

4. A medical device according to claim 1, wherein the communications module is a transceiver control unit.

5. A medical device comprising:
a) a sensing module including a sensing component provided with a housing that includes a first end and a second end;
b) the housing includes a diameter that is less than 1.2 millimeters and encloses a sensing element that is located closer to the first end than the second end;
c) the sensing element includes a first layer and a second layer that are bonded together to form a cavity;
d) the first layer includes a resistive element that is electrically connected to a processing module;
e) the processing module includes an analog-to-digital converter that receives a signal from the resistive element and that is electrically connected to a processing unit; and
f) the processing module is electrically connected to a communications module.

6. A medical device according to claim 5, wherein the first end is shaped to pierce through a facial layer.

7. A medical device according to claim 5, wherein the resistive element is a piezoresistive element

8. A medical device according to claim 5, wherein the communications module is a wireless module.

9. A medical device according to claim 5, wherein the communications module is a transceiver control unit.

10. A medical device according to claim 5, wherein the processing module includes a microcontroller.
